SEQUENCE LISTING (20 sequences)

SEQ ID NO: 1 - DNA, 63 bp, Artificial Sequence
  Synthetic sequence, Aptamer #BLA9-20
atgcggatcccgcgccgtgcagagggagaccggtacccgttcgtgatgcgcgcgaagcttgcg

SEQ ID NO: 2 - DNA, 60 bp, Artificial Sequence
  Synthetic sequence, Aptamer #BLA9-11
atgcggatcccgcgctccgtgagacggcaaattatgggttatatgcgcaagcttcgcgcg

SEQ ID NO: 3 - DNA, 60 bp, Artificial Sequence
  Synthetic sequence, Aptamer #BLA9-34
atgcggatcccgcgcccagaacatcatcccgggttctaatttgtgcgcaagcttcgcgcg

SEQ ID NO: 4 - DNA, 60 bp, Artificial Sequence
  Synthetic sequence, Aptamer #BLA9-3
atgcggatcccgcgccgaggatatagagctacagttaataatgggcgcaagcttcgcgcg

SEQ ID NO: 5 - DNA, 70 bp, Artificial Sequence
  Synthetic sequence, Aptamer #nBPA53
atgcggatcccgcgcccaaaagtttaagcgcgaagatactgttgcgtccacgggccgcgcgaagcttgcg

SEQ ID NO: 6 - DNA, 70 bp, Artificial Sequence
  Synthetic sequence, Aptamer #nBPA38
atgcggatcccgcgcccagaagttaaagcgcgaagatactgttgcgtccacgggccgcgcgaagcttgcg

SEQ ID NO: 7 - DNA, 70 bp, Artificial Sequence
  Synthetic sequence, Aptamer #nBPA20
atgcggatcccgcgccccaattgaagaacgcgcgaagaatataaggtggcctggccgcgcgaagcttgcg

SEQ ID NO: 8 - DNA, 70 bp, Artificial Sequence
  Synthetic sequence, Aptamer #nBPA40
atgcggatcccgcgccccaactgaaggacgcgcgaagaatataaggtggcctggccgcgcgaagcttgcg

SEQ ID NO: 9 - DNA, 70 bp, Artificial Sequence
  Synthetic sequence, Aptamer #nBPA32
atgcggatcccgcgccccaactgagaaacgcgcgaagaatataaggtggcctggccgcgcgaagcttgcg

SEQ ID NO: 10 - DNA, 70 bp, Artificial Sequence
  Synthetic sequence, Aptamer #nBPA37
atgcggatcccgcgccccaacttagaagcgcgcgaagaatataaggtggcctggccgcgcgaagcttgcg

SEQ ID NO: 11 - DNA, 70 bp, Artificial Sequence
  Synthetic sequence, Aptamer #nBPA54
atgcggatcccgcgccccaactgaggaacgcgcgaagaatataaggtggcctggccgcgcgaagcttgcg

SEQ ID NO: 12 - DNA, 70 bp, Artificial Sequence
  Synthetic sequence, Aptamer #nBPA21
atgcggatcccgcgccccaactgaagaacgcgcgaagaatataaggtggcttggccgcgcgaagcttgcg

SEQ ID NO: 13 - DNA, 70 bp, Artificial Sequence
  Synthetic sequence, Aptamer #nBPA50
atgcggatcccgcgccccaactgagaaacgcgcgaagaatataaggtggcttggccgcgcgaagcttgcg

SEQ ID NO: 14 - DNA, 70 bp, Artificial Sequence
  Synthetic sequence, Aptamer #nBPA19
atgcggatcccgcgcccaacggaggactattaagcgcgaaggtggcggtattgtgcgcgcgaagcttgcg

SEQ ID NO: 15 - DNA, 70 bp, Artificial Sequence
  Synthetic sequence, Aptamer #nBPA67
atgcggatcccgcgcccaacggaggactattaagcgcgaagatggcggtattgtgcgcgcgaagcttgcg

SEQ ID NO: 16 - DNA, 60 bp, Artificial Sequence
  Synthetic sequence, Aptamer #nBPA42
atgcggatcccgcgcgcgaagtatacagttaggccgtgtgttggccgcgcgaagcttgcg

SEQ ID NO: 17 - DNA, 29 bp, Artificial Sequence
  Synthetic sequence, Aptamer tBLA9-20v1
cggatcccgcgccgtgcagagggagaccg

SEQ ID NO: 18 - DNA, 31 bp, Artificial Sequence
  Synthetic sequence, Aptamer tBLA9-20v2
gcggatcccgcgccgtgcagagggagaccgc

SEQ ID NO: 19 - DNA, 15 bp, Artificial Sequence
  Synthetic sequence, Forward primer
atgcggatcccgcgc

SEQ ID NO: 20 - DNA, 15 bp, Artificial Sequence
  Synthetic sequence, Reverse primer
cgcaagcttcgcgcg

(19) United States
(12) Patent Application Publication
(10) Pub. No.: US 2022/0267758 A1
(43) Pub. Date: Aug. 25, 2022

(54) GOLD NANOPARTICLE-SELEX BASED SCREENING METHOD FOR TARGET-SPECIFIC APTAMERS

(71) Applicant: INDUSTRY-UNIVERSITY COOPERATION FOUNDATION HANYANG UNIVERSITY, Seoul (KR)

(72) Inventors: Young Pil KIM, Seoul (KR); Eun Song LEE, Seongnam-si (KR); Tae Wuk KIM, Goyang-si (KR)

(73) Assignee: INDUSTRY-UNIVERSITY COOPERATION FOUNDATION HANYANG UNIVERSITY, Seoul (KR)

(21) Appl. No.: 17/634,742

(22) PCT Filed: Aug. 10, 2020
(86) PCT No.: PCT/KR2020/010534

§ 371 Date: Feb. 11, 2022

(30) Foreign Application Priority Data
Aug. 14, 2019 (KR) 10-2019-0099881

(51) Int. Cl. C12N 15/10; C12N 15/115; G01N 33/74

(52) U.S. Cl. CPC C12N 15/1048; C12N 15/115; G01N 33/743; C12N 2310/16

(57) ABSTRACT

Systematic Evolution of Ligand Exponential Enrichment (SELEX) is involved to screen DNA/RNA aptamers that recognize a target molecule (including biomolecules such as nucleic acids, lipids, sugars, proteins, and peptides, hormones, low molecular weight chemical substances, toxic substances, ions, etc.). In general, in order to perform SELEX, a process of fixing a target molecule on a substrate or bead surface is required. In addition, since positive/negative monitoring is not possible in each round of a SELEX process to observe whether an aptamer library is actually well combined with a target substance, whether the SELEX process is proceeded correctly is checked by analyzing aptamers screened through several rounds. In order to remarkably solve these conventional problems and to construct a simpler and easier SELEX technique, the present disclosure provides a new SELEX technique using gold nanoparticles.

### TECHNICAL FIELD

The present disclosure relates to a method for screening an aptamer. The aptamer is a polynucleotide molecule with an ability of being bound specifically to a target substance, and may be used in a protein sensor, a nanosensor, and the like. Existing aptamer screening methods take a lot of time in a screening process, so there is a problem that it takes a long time to obtain an aptamer with a certain efficacy or higher.

### BACKGROUND ART

Systemic Evolution of Ligand Exponential Enrichment (SELEX) is a technique for screening aptamers bound specifically to a target substance, and more particularly to a method for generating and amplifying a polynucleotide library of random sequence and then separating aptamers bound to a target substance.

Positive SELEX is a process of increasing the purity of aptamers having high specificity to a target substance by repeating a SELEX process multiple times.

Negative SELEX is a process of screening aptamers having selective specificity to a target substance by discovering aptamers able to bind to a material having homology with a sample or target substance used in a SELEX process.

### DISCLOSURE

### Technical Problem

Aptamers are similar in use to antibodies in terms of the ability to be bound specifically to a target. However, given that an experimenter can select from an artificially synthesized nucleic acid library for a desired target substance in vitro, the aptamers may be developed for a wide range of targets for which antibodies cannot be developed. In order to select an aptamer, a screening method called SELEX (Nature, 1990, 346, 818-822) is generally used, in which about ten to twenty rounds are repeated and which consists of binding of a target and a library, washing an unbound sequence, dissociating a sequence bound to the target, and amplifying the dissociated sequence. For some targets, a process of modifying the targets and fixing the targets on the surface is necessary for a cleaning process, and after the SELEX round is completed, a post-SELEX process is required to improve the performance of selected aptamers. In addition, monitoring should be performed whenever the 3rd to 5th rounds is completed during the SELEX rounds. Therefore, at least several months are required to select aptamers.

Various methods of modifying SELEX to simplify aptamers selecting process and to select better aptamers have been provided. The representative examples are GO-SELEX (Chem. Commum, 2012, 48, 2071-2073) capable of selecting an aptamer without fixing a target to the surface using graphene oxide, and Click-SELEX (Nature Protocols, 2018, 13, 1153-1180) capable of simplifying a post-SELEX process using click chemistry. However, monitoring is still an important task in SELEX because an experimenter cannot check whether an aptamer is being selected in a desired direction while the SELEX round is in progress. Monitoring is essential to determine the progress of SELEX because there are many variables that can cause failure of SELEX, such as errors in PCR amplifying the library, low purity of the target, and excessive selective pressure in a SELEX process.

### Technical Solution

The present application provides a method for selecting a single-stranded nucleic acid having an ability of being bound to a target substance, the method comprising: i) preparing a gold nanoparticle-single-stranded nucleic acid library; ii) reacting the gold nanoparticle-single-stranded nucleic acid library with a target substance; iii) separating the single-stranded nucleic acid bound to the target substance from the reaction mixture; and iv) determining whether to proceed with an additional reaction through the color index of the reaction mixture.

In addition, the present application provides the method for selecting a single-stranded nucleic acid having an ability of being bound to a target substance, wherein preparing the gold nanoparticle-single-stranded nucleic acid library comprises a) preparing reduced and stabilized gold nanoparticles by using citrate; b) preparing a single-stranded nucleic acid library; c) reacting the gold nanoparticles with the single-stranded nucleic acid library; and d) removing the single-stranded nucleic acid not bound to the gold nanoparticles.

In one aspect, the gold nanoparticles of the present application may have an average diameter of 15 nm to 50 nm.

In addition, the present application provides the step of the iii) separating the single-stranded nucleic acid bound to the target substance from the reaction mixture is performed by obtaining a supernatant after centrifuging the reaction mixture.

In one aspect of the present application, the method for selecting a single-stranded nucleic acid having an ability of being bound to a target substance, further comprises isolating the single-stranded nucleic acid from the target substance after separating the single-stranded nucleic acid bound to the target substance from the reaction mixture. In addition, the present application provides the step of isolating the single-stranded nucleic acid from the target substance is performed by ethanol precipitation.

In another aspect of the present application, the method for selecting a single-stranded nucleic acid having an ability of being bound to a target substance, the step of the iv) determining whether to proceed with an additional reaction through the color index of the reaction mixture comprises a) measuring a color index of the reaction mixture; and b) comparing the color index of the reaction mixture to a standard color index. In addition, the method of the present application further comprises inducing the gold nanoparticles to aggregate prior to measuring the color index of the reaction mixture. Furthermore, the present application provides the step of the inducing the gold nanoparticles to aggregate is carried out adding a salt to the reaction mixture.

In another aspect of the present application, the color index of the reaction mixture is quantified as the ratio of the absorbance values measured at two wavelengths before the gold nanoparticles are aggregated and the absorbance values measured at the same two wavelengths after the gold nanoparticles are aggregated. Furthermore, the present application provides a method for selecting a single strand nucleic acid having an ability of being bound to a target substance, characterized in that the color index of the reaction mixture is E620/E520. A wavelength for measuring the absorbance may be selected differently depending on the size of the gold nanoparticles, and the ratio between absorbances for indicating aggregation change may be used as an inverse number.

In addition, the present application provides a method for selecting a single strand nucleic acid having an ability of being bound to a target substance further comprises performing sequentially separating the single-stranded nucleic acid bound to the target substance from the reaction mixture after determining whether to proceed with an additional reaction through the color index of the reaction mixture. Furthermore, the method according to the present application characterized in that after the single-stranded nucleic acid bound to the target substance is separated, a gold nanoparticle single-stranded nucleic acid library thereof is prepared and a target substance binding single-stranded nucleic acid selecting process is repeated a specific number of times.

In addition, the target substance may comprise Brassinolide or a small molecule material capable of inducing aggregation of gold nanoparticles including Bisphenol A, ions, proteins, nucleic acids, viruses, and microorganisms.

In still another aspect of the present application, the method for selecting a single-stranded nucleic acid having an ability of being bound to a target substance, further comprises a) separating a single-stranded nucleic acid bound to a target substance from the reaction mixture; b) after determining whether to proceed with an additional reaction through the color index of the reaction mixture, preparing a gold nanoparticle-single-stranded nucleic acid library of single-stranded nucleic acids bound to the target substance; c) reacting the gold nanoparticle-single-stranded nucleic acid library with a non-target substance; and d) separating single-stranded nucleic acids that are not bound to a non-target substance from the reaction mixture; wherein the target substance and the non-target substance are different. Furthermore, the present application provides a method for selecting a single-stranded nucleic acid having an ability of being bound to a target substance, characterized in that the target substance is Brassinolide and the non-target substance is B-sitosterol, or the target substance is Bisphenol A and the non-target substance is Bisphenol S.

In addition, the present application provides an isolated single-stranded nucleic acid consisting of one nucleotide sequence selected from SEQ ID NOs: 1 to 4, 17 and 18, preferably SEQ ID NOs: 1 to 4, 17 and 18, and having an ability of being bound to Brassinolide and relatively weak binding to B-sitosterol.

In addition, the present application provides an isolated single-stranded nucleic acid consisting of one nucleotide sequence selected from SEQ ID NOs: 5 to 16, preferably SEQ ID NO: 8, and having an ability of being bound to Bisphenol A and relatively weak binding to Bisphenol S.

In addition, the present application provides a kit for purifying Brassinolide comprising a single-stranded nucleic acid consisting of one or more nucleotide sequences selected from SEQ ID NOs: 1, 17 and 18.

In another aspect, the present application provides a kit for purifying Brassinolide comprising a single-stranded nucleic acid consisting of one nucleotide sequences selected from SEQ ID NOs: 1 to 4, 17 and 18, preferably SEQ ID NOs: 1, 17 and 18.

### Advantageous Effects

The present invention is to solve the above problems, and does not require complicated measuring equipment, and is a method that can check the progress of SELEX in a short time simply by changing the color of nanoparticles. Method for measurement through colorimetry of gold nanoparticles (e.g. DNA detection, protein detection, heavy metal ion detection, immunoassay, etc.) have been widely performed, but a method for monitoring an aptamer selecting process through color change of nanoparticles has never been reported. In this technique, in a case where a binding force between ssDNA and the target substance is high, when the gold nanoparticles meet a salt, the surface of the gold nanoparticles is denatured, and aggregation of the gold nanoparticles occurs. During the aggregation process, the gold nanoparticles induce a color change. This is a phenomenon that occurs because surface plasmon and a degree of light scattering vary according to the size of the nanoparticles. That is, normal gold nanoparticles have a red-brown color, but when the gold particles aggregate, the particles increase in size and scatter a larger wavelength, so that the color of a solution in which the gold nanoparticles are dissolved changes to purple, blue, gray, etc. depending on the size of the gold nanoparticles. On the other hand, when the binding force between ssDNA and the target is weak, the aggregation of the gold nanoparticles is inhibited and the color changes less, and thus, the binding force between the ssDNA pool and the target in the SELEX round may be rapidly and quickly analyzed through the color change. The change in the color of the gold nanoparticles in a solution may be quantified simply by simple absorption analysis, and since monitoring can be easily performed by adding a salt at the end of each round, it is possible to evaluate the SELES process more effectively compared to existing methods.

As such, if the affinity of the gold nanoparticles to a single-stranded DNA and the aggregation of the gold nanoparticles due to a salt are used, it is possible to analyze and monitor a SELEX process more quickly and conveniently. In particular, a separate monitoring process is not required, and the progress is determined only through the color change of the gold nanoparticles, and thus, the aptamer selecting process may be performed flexibly and experimenter-friendly compared to the existing SELEX. In addition, it is possible to select an aptamer as the original shape of a desired target in a labeling-free method that does not require chemical modification of the target for SELEX. In doing so, not only the selection of an aptamer for a substance of interest may be simplified, but it is possible to use a selected aptamer for target imaging, medical diagnosis, toxicity sensing in the environment or food field, and target-specific drug delivery therapy, thereby expecting development.

### DESCRIPTION OF DRAWINGS

FIG. 1 is a schematic diagram illustrating a method for selecting a single-stranded nucleic acid according to the present application.

FIG. 2 is Example 1 of a target substance binding single-stranded nucleic acid selecting process.

FIG. 3 is an exemplary preparing process for gold nanoparticle single-stranded nucleic acid library.

FIG. 4 is Example 2 of the target substance binding single-stranded nucleic acid selecting process.

FIG. 5 is Example 3 of the target substance binding single-stranded nucleic acid selecting process.

FIG. 6 is Example 4 of the target substance binding single-stranded nucleic acid selecting process.

FIG. 7 is Example 5 of the target substance binding single-stranded nucleic acid selecting process.

FIG. 8 is an example of a single-stranded nucleic acid selection method according to the present application, including a target substance binding single-stranded nucleic acid selecting process and a non-target substance non-binding single-stranded nucleic acid selecting process.

FIG. 9 is a color index (E620/E520) measured in the middle of the selection process for selecting a single-stranded nucleic acid to be bound to Brassinolide.

FIG. 10 is a photograph of a reaction mixture taken in the middle of the selection process for selecting a single-stranded nucleic acid to be bound to Brassinolide.

FIG. 11 is a color index (E620/E520) measured in the middle of the selection process for a selecting single-stranded nucleic acid to be bound to Bisphenol A.

FIG. 12 is a photograph of a reaction mixture taken in the middle of the selection process for selecting a single-stranded nucleic acid to be bound to Bisphenol A.

FIG. 13 illustrates a secondary structure of a single-stranded nucleic acid BLA8-20 to be bound to Brassinolide.

FIG. 14 is a graph showing the measurement of binding force between a single-stranded nucleic acid selected by a method according to the present application and Brassinolide or non-target substances.

FIG. 15 is a photograph of a reaction mixture obtained by reacting a single-stranded nucleic acid selected, by a method according to the present application, with Brassinolide and non-target substances.

FIG. 16 illustrates a secondary structure of a single-stranded nucleic acid nBPA40 to be bound to Bisphenol A.

FIG. 17 is a measurement of a binding force between a single-stranded nucleic acid selected by a method according to the present application and Bisphenol A or non-target substances.

FIG. 18 is a photograph of a reaction mixture obtained by reacting a single-stranded nucleic acid selected by a method according to the present application with Bisphenol A or non-target substances.

FIG. 19 is a comparison of measurement of a binding force between a single-stranded nucleic acid nBPA40 and a previously published aptamer with respect to Bisphenol A.

FIG. 20 is a photograph of a reaction mixture obtained by reacting a single-stranded nucleic acid nBPA40 and a previously published aptamer with Bisphenol A.

FIG. 21 is a result of comparing the sequence similarity between a single-stranded nucleic acid nBPA40 and aptamer previously published aptamer.

FIG. 22 illustrates a single-stranded nucleic acid BLA9-20 and an improved single-stranded nucleic acid manufactured by truncating the same.

FIG. 23 is a result of measuring a binding force of tBLA-v1 and tBLA-v2 with Brassinolide.

FIG. 24 is a measurement of the change in the secondary structure after binding of tBLA-v1 and Brassinolide.

FIG. 25 is a measurement of the change in the secondary structure after binding of tBLA-v2 and Brassinolide.

FIG. 26 is an example illustrating a method for detecting Brassinolide using an aptamer.

FIG. 27 is an experimental result for quantifying the amount of Brassinolide from Arabidopsis extracts containing different concentrations of Brassinolide.

### MODES OF THE INVENTION

### Definition

As used herein, the terms “react”, “reacting”, and “to react” refer to adjoining reactants through mixing, addition, etc. so that they can interact, and/or a phenomenon by this. A reaction according to the present application does not matter whether the result is a physical change or a chemical change of a reactant.

The terms “nucleic acid” and “single-stranded nucleic acid” in the present application follow commonly known definitions. In particular, since the method for selecting a nucleic acid according to the present application and the nucleic acid selected thereby are the concepts that can be extended regardless of the type of the nucleic acid, the nucleic acid of the present application is intended to include not just DNA and RNA, but also all those that can be understood at the current technology level.

As used herein, the term “nucleic acid library” refers to a collection of at least two different nucleic acids. As used herein, the term “single-stranded nucleic acid library” refers to a collection of at least two different single-stranded nucleic acids. The nucleic acid library according to the present application may be in any form, such as a form in which the nucleic acid is contained in a microorganism, a form contained in a special formulation (micelle, liposome, etc.), a form dispersed without a special formulation, and the like.

The “target substance” refers to the specific substance when selecting a single-stranded nucleic acid having binding to a specific substance, which is the objective of the present application. The “non-target substance” refers to a substance intended not to be bound to a single-stranded nucleic acid in selecting of the single-stranded nucleic acid. The target substance and the non-target substance may be in any form of small molecules, ions, proteins, nucleic acids, viruses, and microorganisms.

As used herein, the terms “reaction mixture,” “reaction combination,” and the like are intended to refer to the state of the mixture in the description of the steps of a specific method. That is, these are terms used to refer to a time-series product itself made by a reaction, step, or the like that has already been carried out before a corresponding step, and such terms are not intended to be limited by a specific composition or configuration.

Preparation of Gold Nanoparticle-Single-Stranded Nucleic Acid Library

Outline

The present application provides a method for preparing a gold nanoparticle single-stranded nucleic acid library. The “gold nanoparticle single-stranded nucleic acid library” is formed by an adjacent positional relationship between gold nanoparticles and single-stranded nucleic acids, and refers to a collection of at least two combinations of the gold nanoparticles and single-stranded nucleic acids.

In one embodiment, the method for preparing a gold nanoparticle single-stranded nucleic acid library may include reacting gold nanoparticles with a single-stranded nucleic acid library. The gold nanoparticles and the single-stranded nucleic acid library are bound to each other through a reaction to form a gold nanoparticle single-stranded nucleic acid library. In this case, the binding is any of binding through a physical force such as an electrostatic force, chemical binding, and the like. In one example, the gold nanoparticle single-stranded nucleic acid library may be a single-stranded nucleic acid adsorbed around gold nanoparticles. In another example, in the gold nanoparticle single-stranded nucleic acid library, a single-stranded nucleic acid may be chemically bound to gold nanoparticles.

Preparation of Gold Nanoparticle

The method for preparing a gold nanoparticle single-stranded nucleic acid library of the present application may comprise preparing gold nanoparticles. In this case, the method for preparing gold nanoparticles may be a conventionally known method for preparing gold nanoparticles.

In one embodiment, the gold nanoparticles of the present application may be stabilized with citrate to have affinity to ssDNA. In one embodiment, the gold nanoparticles of the present application may have an average diameter of 15 nm to 50 nm. The gold nanoparticles of the present application may be prepared by reduction and stabilization with citrate.

Preparation of Single-Stranded Nucleic Acid Library

The method for preparing a gold nanoparticle single-stranded nucleic acid library of the present application may include preparing a single-stranded nucleic acid library. In this case, a method for preparing a single-stranded nucleic acid library may be performed by a commonly known method for preparing a nucleic acid library.

In one embodiment, the single-stranded nucleic acid library of the present application may be prepared by a DNA synthesizer, error-prone PCR, mutagenesis, and the like. In one embodiment, the single-stranded nucleic acid subjected to the single-stranded nucleic acid selecting process according to the present application may be used as a single-stranded nucleic acid library.

Additional Process

The method for preparing a gold nanoparticle single-stranded nucleic acid library according to the present application may include an additional process in addition to the above-mentioned processes.

In one embodiment, the preparation method may include reacting gold nanoparticles with a single-stranded nucleic acid library and then removing a single-stranded nucleic acid not bound to the gold nanoparticles. Some single-stranded nucleic acids may not bind well to gold nanoparticles by nature. When such single-stranded nucleic acids not bound to the gold nanoparticles are included in a library, it may lead to an erroneous result in the following single-stranded nucleic acid selecting process. For example, in the following single-stranded nucleic acid selecting process, the single-stranded nucleic acids not bound to the gold nanoparticles do not react with a target substance, but are separated in the form of single-stranded nucleic acids and become impurities. In addition, in the process of monitoring through the following colorimetric reaction, the single-stranded nucleic acids are unintentionally dissociated from the gold nanoparticles and act as a cause of the color index error of the mixture.

Therefore, by applying (such as centrifugation) a physical impact, which may occur during the selection process, to the library in advance, it is possible to remove single-stranded nucleic acids that may act as a cause of error. In one example thereof, removing the single-stranded nucleic acid not bound to the gold nanoparticles may include discarding a supernatant obtained from centrifugation of the reaction product and obtaining the remainder. In this case, the centrifugation may be performed at 3000 to 9000 g. Preferably, the centrifugation may be performed at 6500 g. Alternatively, the centrifugation may be performed for five to twenty minutes. Preferably, the centrifugation may be performed for ten minutes. Alternatively, removing the single-stranded nucleic acid not bound to the gold nanoparticles may include performing a predetermined number of times an operation of discarding a supernatant obtained from centrifugation of the reaction product.

Reaction of Gold Nanoparticles-Single-Stranded Nucleic Acid Library with Target Substance

By reacting the gold nanoparticle single-stranded nucleic acid library with a target substance, a single-stranded nucleic acid bound to the target substance (hereinafter, referred to as “target substance binding single-stranded nucleic acid”) may be detected. This is because the single-stranded nucleic acid bound to the target substance changes its interaction with the gold nanoparticles (for example, the single-stranded nucleic acid may be separated). In one embodiment, the reaction may comprises mixing the gold nanoparticle single-stranded nucleic acid library and the target substance.

Separating Target Substance Binding Single-Stranded Nucleic Acid

In this section, a method for separating a target substance binding single-stranded nucleic acid, which caused a reaction, after a gold nanoparticle single-stranded nucleic acid library reacts with a target substance will be described.

As described above, the single-stranded nucleic acid bound to the target substance changes its interaction with the gold nanoparticles, and the single-stranded nucleic acid that does not react with the target substance remains bound to the gold nanoparticles. Accordingly, it is possible to separate these single-stranded nucleic acids by applying a physical impact or chemical treatment to the reaction mixture.

In one embodiment, the target substance binding single-stranded nucleic acid may be separated by applying a physical impact to the reaction mixture. In one example, the reaction mixture may be phase-separated to separate a target substance binding single-stranded nucleic acid. The separated single-stranded nucleic acid has a smaller mass than that of a gold nanoparticle single-stranded nucleic acid structure. In a specific example, separating the single-stranded nucleic acid bound to the target substance may include obtaining a supernatant after centrifuging the reaction mixture.

Isolating Target Substance Binding Single-Stranded Nucleic Acid

The method for selecting a single-stranded nucleic acid having an ability of being bound to a target substance according to the present application may include selectively isolating the target substance binding single-stranded nucleic acid. The isolating means removing the single-stranded nucleic acid from the single-stranded nucleic acid bound to the target substance and then isolating the single-stranded nucleic acid. This may be performed by removing the single-stranded nucleic acid bound to the target substance, separating the single-stranded nucleic acid from the target substance and then isolating the single-stranded nucleic acid.

In this case, the isolation of the single-stranded nucleic acid may be performed by commonly used methods. In one embodiment, isolating the separated single-stranded nucleic acid may comprise performing ethanol precipitation.

Amplification of Target Substance Binding Single-Stranded Nucleic Acid

The method for selecting a single-stranded nucleic acid having an ability of being bound to a target substance according to the present application may comprise selectively amplifying a target substance binding single-stranded nucleic acid. In one embodiment, the single-stranded nucleic acid bound to the target substance may be separated as described above and then amplified in order to increase the purity of an amplified sequence.

For the amplification of the single-stranded nucleic acid, any commonly used methods such as PCR and artificial synthesis of nucleic acids may be used.

Properties of Gold Nanoparticles: Colorimetric Reaction and Monitoring of Selection Process

As described below, gold nanoparticles have useful properties for monitoring the selection process according to the present application.

In one embodiment, the method for selecting a single-stranded nucleic acid having an ability of being bound to a target substance according to the present application may include colorimetric monitoring.

Aggregation and Colorimetric Reaction of Gold Nanoparticles

Gold nanoparticles have a characteristic that physical properties thereof change as the light absorption spectrum changes according to the size of the particles. Typically, as the particles increases in size, the mixture containing gold nanoparticles changes from red to purple. Gold nanoparticles have been used in colorimetric sensor technology.

The reason that the size of the particles changes is that the gold nanoparticles aggregate. The gold nanoparticle single-stranded nucleic acid structure according to the present application has electric charges placed on the surface thereof and maintained in a dispersed form by a repulsive force. When the single-stranded nucleic acid is separated from the gold nanoparticle single-stranded nucleic acid structure and the electronic charges on the surface of the gold nanoparticles are overcome, the gold nanoparticles are aggregated with each other. Typically, it is known that salt-induced aggregation occurs when a salt is added to a gold nanoparticle solution. The physical properties of the gold nanoparticles may be effectively utilized for monitoring the selection process in the method for selecting a single-stranded nucleic acid according to the present application.

Colorimetric Index of Mixture Containing Gold Nanoparticles

The “color index” refers to a number that can express the optical properties of a specific object (e.g., wavelength, refractive index, frequency, energy, absorbance, or reflectance, etc.). A mixture containing gold nanoparticles produced in the selection process according to the present application has a specific color. That is, depending on an average diameter of the gold nanoparticles included in the mixture, the color of the gold nanoparticles changes from red to purple (as the gold nanoparticle change from a small size to a large size). The average diameter of the gold nanoparticles may be changed by aggregation of the gold nanoparticles.

In one embodiment, the method for selecting a single-stranded nucleic acid having an ability of being bound to a target substance according to the present application may further include inducing Au nanoparticles to aggregate. Further, inducing the Au nanoparticles to aggregate may be performed prior to measuring the color index of the reaction mixture.

In one example, a salt may be added to the mixture to induce salt-induced aggregation. In this case, if the gold nanoparticles are in the form of a gold nanoparticle single-stranded nucleic acid structure, the salt and the gold nanoparticles do not react and thus do not properly aggregate. However, if the gold nanoparticles are separated from the single-stranded nucleic acid (for example, by reaction with a target substance), the salt and the gold nanoparticles react and the gold nanoparticles are aggregated. The degree of aggregation may depend on how much the single-stranded nucleic acid is separated. The selection process may be monitored by checking the color index that changes with the size of the aggregated particles.

In one example, the color index of the mixture may be absorbance of the mixture. Furthermore, the color index of the mixture may be a ratio of specific wavelength values in an absorption curve. In one example, the color index of the mixture may be extracted from a photograph of the mixture.

Determination of Whether to Proceed with Additional Process Through Color Index

One) Outline

The present application provides a method for determining whether to proceed with an additional process through a color index of a reaction mixture during the single-stranded nucleic acid selecting process according to the present application. The selection process may be monitored by the above-described properties of the gold nanoparticles. In addition, in doing so, it is possible to determine whether an additional process should be performed. For example, when it is determined that single-stranded nucleic acids are not selected as much as desired, an additional selection process may be performed. Gold nanoparticles have the advantage in that optically monitoring the selection process is possible without any manipulation.

According to the selection process of the present application, the more the single-stranded nucleic acids contained in the gold nanoparticle single-stranded nucleic acid library reacts with the target substance, the more the single-stranded nucleic acids are separated from the gold nanoparticles. Since the surface of the gold nanoparticles is more exposed, the gold nanoparticles are well aggregated. In this case, the color of the mixture changes from red to purple when the particles change from a small size to a large size. That is, as the binding of the single-stranded nucleic acid to the target is improved, the color of the aggregation-induced mixture becomes more like purple.

2) Sequence in the Process

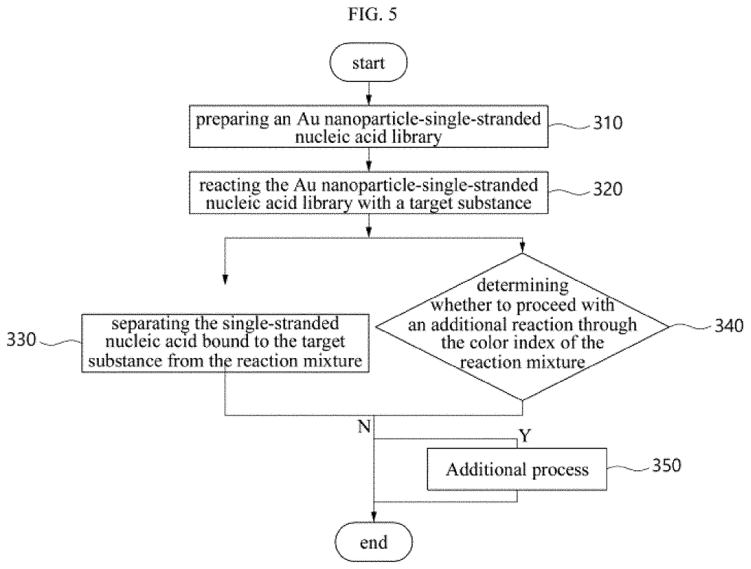

In one embodiment, a method for selecting a single-stranded nucleic acid having an ability of being bound to a target substance may further comprises preparing a gold nanoparticle single-stranded nucleic acid library; and determining whether to proceed with an additional process through a color index of the reaction mixture after reacting the gold nanoparticle single-stranded nucleic acid library with a target substance. In this case, determining whether to proceed with the additional process may be performed before to or after separating the single-stranded nucleic acid bound to the target substance from the reaction mixture. In one example, separating the single-stranded nucleic acid bound to the target substance from the reaction mixture may be performed after determining whether to proceed with the additional process through the color index of the reaction mixture. FIG. 5 shows a flowchart of an example of a single-stranded nucleic acid selecting process including determining whether to proceed with an additional reaction.

In one embodiment, prior to determining whether to proceed with the additional process through the color index of the reaction mixture according to the present application, inducing the gold nanoparticles to aggregate may be performed. In one example, inducing the gold nanoparticles to aggregate may include adding a salt to the reaction mixture. In this case, the salt may be NaCl.

3) How to Determine

Through the color index of the reaction mixture, whether to proceed with the additional process may be determined. This is because it is possible to know qualitatively and quantitatively through the color index how much the selection process has progressed. In one embodiment, determining whether to proceed with the additional process through the color index of the reaction mixture comprises measuring the color index of the reaction mixture; and comparing the color index of the reaction mixture to a reference color index.

The color index of a reactant which serves as the basis of the determination may be selected from commonly known color indices. In one example, the color index of the reaction mixture may be absorbance. Furthermore, the color index of the reaction mixture may be a ratio of a specific wavelength value of an absorption curve. Furthermore, the color index of the reaction mixture may be a ratio of absorbance at 620 nm to absorbance at 520 nm (hereinafter, the ratio is E620/E520). In this case, a wavelength for measuring the absorbance may be selected differently depending on the size of the gold nanoparticles, and the ratio between absorbance values indicating aggregation change may be used as an inverse number. In one example, the color index of the reaction mixture may be derived from a photograph of the reaction mixture. For example, the color index of the reaction mixture may be data that can be processed computationally, such as an RGB code of the color of the reaction mixture. In one example, in determining whether to proceed with the additional process, color indices of mixtures of two or more types may be used as a reference.

In a case where the determination process includes measuring the color index of the reaction mixture, the measurement is as follows. In one example, measuring the color index of the reaction mixture may be measuring absorbance of the reaction mixture. Alternatively, measuring the color index of the reaction mixture may be measuring an absorption curve of the reaction mixture. In one example, measuring the color index of the reaction mixture may be taking a picture of the reaction mixture.

In a case where the determination process includes comparing a color index of the reaction mixture to a standard color index, the comparison is as follows. The standard color index refers to the absolute value of the color index for evaluating whether the additional process is required. In one example, the color index of the reaction mixture may be greater than or equal to the standard color index. In one example, the color index of the reaction mixture may be less than or equal to the standard color index. In one example, the color index of the reaction mixture may need to be the same as the standard color index. The following are examples. As the binding between the target substance and the single-stranded nucleic acid is improved, the reaction mixture becomes more like purple and the value of E620/E520 increases. Therefore, E620/E520 of the reaction mixture may be set to be greater than or equal to a specific value of E620/E520 so that the binding of the single-stranded nucleic acid is greater than or equal to a specific value. Furthermore, when E620/E520 of the reaction mixture is less than or equal to a specific value of E620/E520, the selection process may be repeated. In one example, the standard color index may be a color index before the gold nanoparticles are aggregated. Furthermore, the standard color index may be a ratio of absorbance values measured at two wavelengths before the gold nanoparticles are aggregated.

4) Additional Process

In this section, an additional process will be described, the additional process that is performed according to the determination process.

In one embodiment, the additional process may be repeating a target substance binding single-stranded nucleic acid selecting process with respect to the same target substance. For example, when it is determined that the binding of the single-stranded nucleic acid is not sufficiently improved, the binding of the single-stranded nucleic acid may be improved by repeating the selection process.

In one embodiment, the additional process may be performing a target substance binding single-stranded nucleic acid selecting process with respect to different target substances. For example, when it is desired to prepare a single-stranded nucleic acid having an ability of being bound to two or more target substances, the above-described additional process may be employed.

In another embodiment, the additional process may be performing a non-target substance non-binding single-stranded nucleic acid selecting process with respect to a non-target substance.

Target Substance Binding Single-Stranded Nucleic Acid Selecting Process

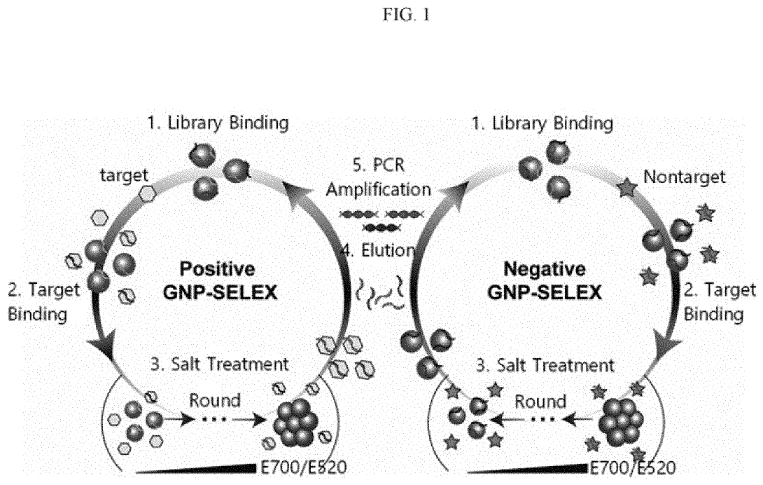

The present application provides a process for selecting a single-stranded nucleic acid having an ability of being bound to a target substance. The above is to describe one operation as a configuration of the selection process. In this section, a target substance binding single-stranded nucleic acid selecting process which is a combination of the above-described processes will be described. The “target substance binding single-stranded nucleic acid selecting process” is a process according to the present application and refers to a process for selecting a single-stranded nucleic acid having an ability of being bound to a specific target substance. The left side of FIG. 1 shows an example of the target substance binding single-stranded nucleic acid selecting process.

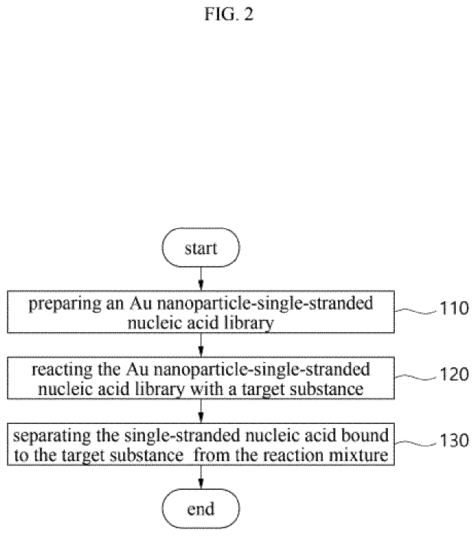

The target substance binding single-stranded nucleic acid selecting process according to the present application may comprise preparing a gold nanoparticle single-stranded nucleic acid library; reacting the gold nanoparticle single-stranded nucleic acid library with a target substance; and separating a single-stranded nucleic acid bound to the target substance from a reaction mixture. The basics of the single-stranded nucleic acid selecting process using gold nanoparticles may be seen in FIG. 2.

In one embodiment, the target substance binding single-stranded nucleic acid selecting process according to the present application may further comprise isolating the single-stranded nucleic acid from the target substance after separating the single-stranded nucleic acid bound to the target substance from the reaction mixture.

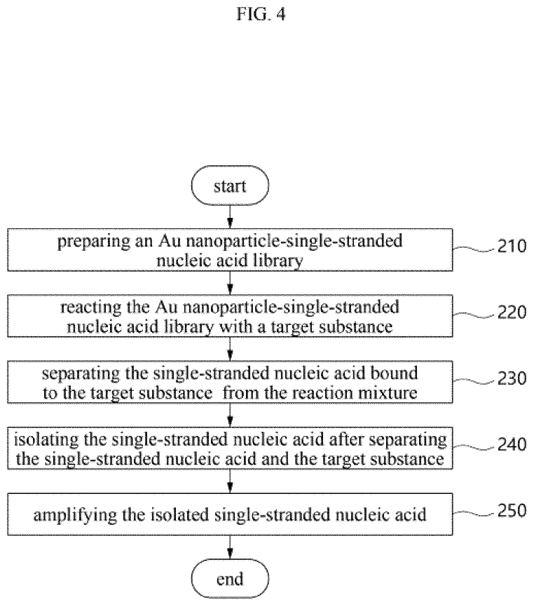

In one embodiment, the target substance binding single-stranded nucleic acid selecting process according to the present application may further comprise amplifying the separated single-stranded nucleic acid after isolating the single-stranded nucleic acid bound to the target substance. An exemplary single-stranded nucleic acid selecting process may be seen in FIG. 4.

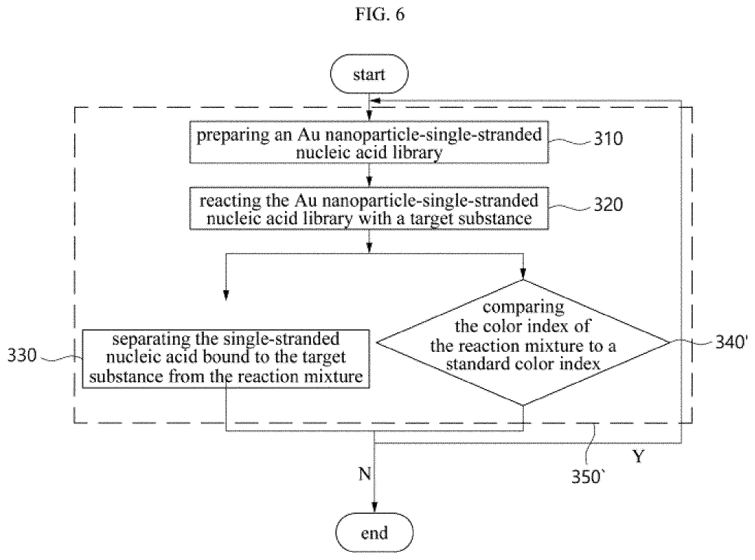

In one embodiment, the target substance binding single-stranded nucleic acid selecting process according to the present application may further comprise determining whether to proceed with an additional process through a color index of a reaction mixture. An exemplary single-stranded nucleic acid selecting process (300) may be seen in FIG. 5. In this case, an example in which the additional process is repeating a target substance binding single-stranded nucleic acid selecting process with respect to the same target substance (350′) may be seen in FIG. 6.

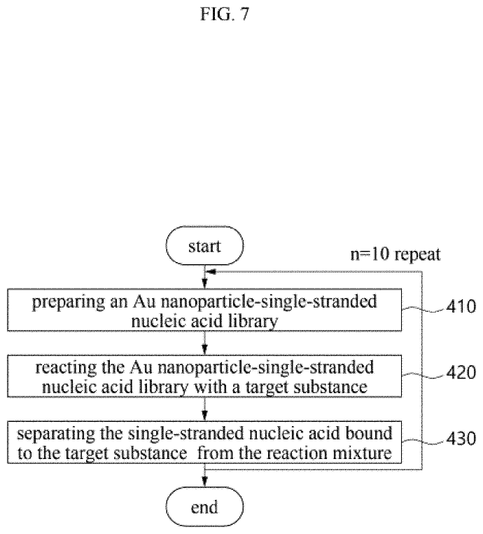

In one embodiment, the target substance binding single-stranded nucleic acid selecting process according to the present application may be repeated a specific number of times. As the selection process is repeated, the binding of the single-stranded nucleic acid to the target substance will be improved. An exemplary single-stranded nucleic acid selecting process may be seen in FIG. 7.

In one embodiment, the target substance of the present application may be Brassinolide or Bisphenol A.

Non-Target Substance Non-Binding Single-Stranded Nucleic Acid Selecting Process

Outline

In some cases, it is a compound having a structure similar to that of a target substance. For example, environmental hormones have a structure similar to that of endocrine hormones in human body. When such a target substance binding single-stranded nucleic acid is selected, the single-stranded nucleic acid may be likely to have binding to a similar structure (e.g., endocrine hormones). In some cases, this may adversely affect the purity resulting from a screening process and cause side effects of a treatment method.

Accordingly, it may be preferable to select a single-stranded nucleic acid that has an ability of being bound to a target substance but does not have binding to a similar non-target substance. In general, this problem is solved through negative SELEX. The non-target substance may be different from the target substance.

The present application provides a method for selecting a single-stranded nucleic acid are not bound to a non-target substance in a method for selecting a single-stranded nucleic acid based on gold nanoparticles. The “non-target substance non-binding single-stranded nucleic acid selecting process” is a process according to the present application and refers to a process of selecting a single-stranded acid not bound to a specific non-target substance. The non-target substance non-binding single-stranded nucleic acid selecting process is performed similarly to the target substance binding single-stranded nucleic acid selecting process that has been comprehensively described above. The target substance binding single-stranded nucleic acid selecting process includes separating a single-stranded nucleic acid bound to a target substance from a reaction mixture. The non-target substances non-binding single-stranded nucleic acid selecting process is different in that separating a single-stranded nucleic acid not bound to a non-target substance from the reaction mixture is included. The right side of FIG. 1 shows an example of a non-target substance non-binding single-stranded nucleic acid selecting process.

Preparation of Gold Nanoparticle-Single-Stranded Nucleic Acid Library

In the non-target substance non-binding single-stranded nucleic acid selecting process, preparation of a gold nanoparticle single-stranded nucleic acid library applies the above descriptions. For example, a single-stranded nucleic acid in the gold nanoparticle single-stranded nucleic acid library may be a single-stranded nucleic acid selected by the target substance binding single-stranded nucleic acid selecting process.

Reaction of Gold Nanoparticles-Single-Stranded Nucleic Acid Library with Non-Target Substance

In the non-target substance non-binding single-stranded nucleic acid selecting process, reaction between a gold nanoparticle-single-stranded nucleic acid library and a non-target substance applies to the above descriptions.

Separation of Non-Target Substances Non-Binding Single-Stranded Nucleic Acid

Unlike the above-described separation of a single-stranded nucleic acid bound to a target substance, it is preferable to separate a non-binding single-stranded nucleic acids not bound to a non-target substance in the selection process of this section. Therefore, it is necessary to separate the single-stranded nucleic acid bound to the non-target substance by applying a physical impact or chemical treatment to the reaction mixture, and to separate the remaining single-stranded nucleic acid in the form of a gold nanoparticles-single-stranded nucleic acid.

In one embodiment, it is possible to separate the non-target substance non-binding single-stranded nucleic acid after applying a physical impact to the reaction mixture. In one example, the reaction mixture may be phase-separated to separate the non-target substance non-binding single-stranded nucleic acid. The separated single-stranded nucleic acid has a smaller mass than that of a gold nanoparticle-single-stranded nucleic acid structure. In a specific example, separating a single-stranded nucleic acid not bound to a non-target substance may include centrifuging the reaction mixture and then obtaining the remainder except for a supernatant.

Isolation/Amplification of Non-Target Substances Non-Binding Single-Stranded Nucleic Acid

In the non-target substance non-binding single-stranded nucleic acid selecting process, the isolation and amplification of the non-target substance non-binding single-stranded nucleic acid apply the above descriptions.

However, the difference lies in that the process of isolating the single-stranded nucleic acid non-binding to the non-target substance is detaching the single-stranded nucleic acid from the gold nanoparticles. The non-target substance non-binding single-stranded nucleic acid selecting process according to the present application may further comprise separating the single-stranded nucleic acid not bound to the non-target substance from the reaction mixture, separating the single-stranded nucleic acid from the gold nanoparticles, and then isolating the single-stranded nucleic acid. In one example, the single-stranded nucleic acid and the gold nanoparticles may be separated by heating the reaction mixture. Furthermore, the single-stranded nucleic acid and the gold nanoparticles may be separated by heating the reaction mixture at 95° C.

Monitoring Through Colorimetric Reaction of Gold Nanoparticles

In the non-target substance non-binding single-stranded nucleic acid selecting process, a monitoring method of the process applies the above-described descriptions. However, in this selection process, as the process progresses, a binding force of the single-stranded nucleic acid decreases, so that the binding between the single-stranded nucleic acid and the gold nanoparticles will be maintained. Therefore, unlike the target substance binding single-stranded nucleic acid selecting process, in this separation process, the color of the reaction mixture changes from purple to red as the binding force between the single-stranded nucleic acid and the non-target substance decreases.

As the binding between the non-target substance and the single-stranded nucleic acid decreases, the color of the reaction mixture becomes more like red and the value of E620/E520 decreases. In one example, E620/E520 of the reaction mixture may be set to be less than or equal to a specific value of E620/E520 so that the binding of the single-stranded nucleic acid is less than or equal to a specific value. Furthermore, if E620/E520 of the reaction mixture is greater than or equal to the specific value of E620/E520, the selection process may be repeated again.

Entire Process

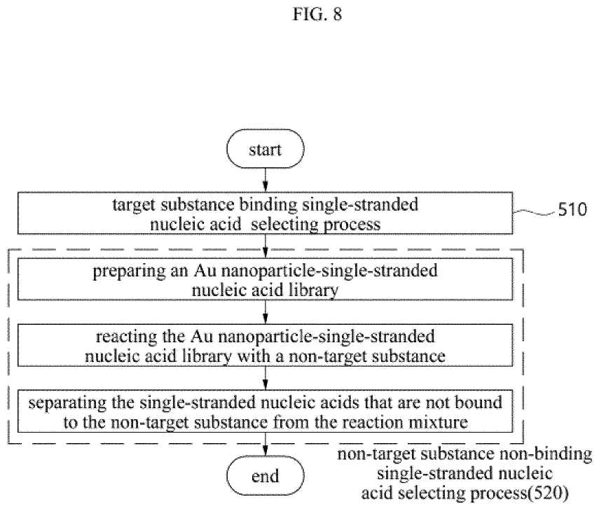

The non-target substance non-binding single-stranded nucleic acid selecting process according to the present application may comprise preparing a gold nanoparticle single-stranded nucleic acid library; reacting the gold nanoparticle single-stranded nucleic acid library with a non-target substance; and separating a single-stranded nucleic acid not bound to the non-target substance from a reaction mixture. The basics of the single-stranded nucleic acid selecting process using gold nanoparticles (520) may be found in FIG. 8.

In one embodiment, the non-target substance non-binding single-stranded nucleic acid selecting process according to the present application may further comprise separating the single-stranded nucleic acid not bound to the non-target substance from the reaction mixture, and separating the single-stranded nucleic acid from the gold nanoparticles, and then isolating the separated single-stranded nucleic acid.

In one embodiment, the non-target substance non-binding single-stranded nucleic acid selecting process according to the present application may further include separating the single-stranded nucleic acid not bound to the non-target substance and then amplifying the separated single-stranded nucleic acid.

In one embodiment, the non-target substance non-binding single-stranded nucleic acid selecting process according to the present application may further include determining whether to proceed with an additional process through a color index of the reaction mixture.

In one embodiment, the non-target substance non-binding single-stranded nucleic acid selecting process according to the present application may be repeated a specific number of times. As the selection process is repeated, the binding of the single-stranded nucleic acid to the non-target substance may become weak.

In one embodiment, the non-target substance of the present application may be B-sitosterol or Bisphenol S.

Method for Selecting Single-Stranded Nucleic Acids According to the Present Application

The present application provides a method for selecting a single-stranded nucleic acid. In the above descriptions, a process for selecting a single-stranded nucleic acid having binding to a target substance or a process for selecting a single-stranded nucleic acid not having binding to a non-target substance have been described as a method for selecting a single-stranded nucleic acid. In this section, there is provided a method for selecting a desired single-stranded nucleic acid by combining these processes. For example, by serially performing the target substance binding single-stranded nucleic acid selecting process and the non-target substance non-binding single-stranded nucleic acid selecting process according to the present application, it is possible to obtain a single-stranded acid that has binding to a target substance and non-binding to a non-target substance. The “target substance binding single-stranded nucleic acid selecting process” and “non-target substance non-binding single-stranded nucleic acid selecting process” mentioned below include all embodiments and examples described above.

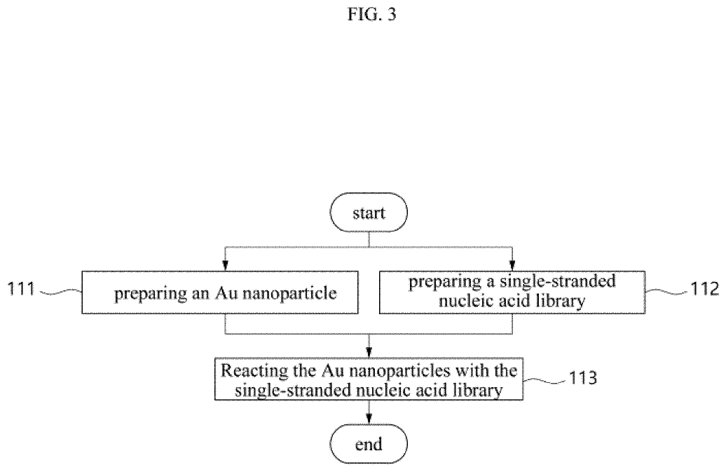

In one embodiment, the method for selecting a single-stranded nucleic acid according to the present application may comprise a target substance binding single-stranded nucleic acid selecting process. In one example, the method for selecting a single-stranded nucleic acid according to the present application may include a target substance binding single-stranded nucleic acid selecting process with respect to two or more target substances. For example, when two target substances are selected, the method for selecting a single-stranded nucleic acid according to the present application may comprise a target substance binding single-stranded nucleic acid selecting process with respect to a first target substance, and a target substance binding single-stranded nucleic acid selecting process with respect to a second target substance. FIGS. 2 to 7 illustrate examples including a target substance binding single-stranded nucleic acid selecting process.

In one embodiment, the method for selecting a single-stranded nucleic acid according to the present application may comprise a non-target substance non-binding single-stranded nucleic acid selecting process. In one example, the method for selecting a single-stranded nucleic acid according to the present application may include a non-target substance non-binding single-stranded nucleic acid selecting process with respect to two or more non-target substances. For example, when two non-target substances are selected, the method for selecting a single-stranded nucleic acid according to the present application may include a non-target substance non-binding single-stranded nucleic acid selecting process with respect to a first non-target substance, and a non-target substance non-binding single-stranded nucleic acid selecting process with respect to a second non-target substance.

In one embodiment, the method for selecting a single-stranded nucleic acid according to the present application may comprise a target substance binding single-stranded nucleic acid selecting process and a non-target substance non-binding single-stranded nucleic acid selecting process. In doing so, it is possible to select a single-stranded nucleic acid that has binding to a target substance while having weak binding to a non-target substance. In one example, the non-target substance non-binding single-stranded nucleic acid selecting process may be performed after the target substance binding single-stranded nucleic acid selecting process is performed. Furthermore, the non-target substance non-binding single-stranded nucleic acid selecting process may be performed after a single-stranded nucleic acid bound to the target substance is separated in the target substance binding single-stranded nucleic acid selecting process. Moreover, the target substance binding single-stranded nucleic acid selecting process and the non-target substance non-binding single-stranded nucleic acid selecting process may be alternately performed. For example, after a target substance binding single-stranded nucleic acid selecting process is performed, a non-target substance non-binding single-stranded nucleic acid selecting process may be performed and then the target substance binding single-stranded nucleic acid selecting process may be performed again. In one example, the target substance may be Brassinolide and the non-target substance may be B-sitosterol. In one example, the target substance may be Bisphenol A and the non-target substance may be Bisphenol S. FIG. 8 illustrates examples including a target substance binding single-stranded nucleic acid selecting process (510) and a non-target substance non-binding single-stranded nucleic acid selecting process (520).

In the present application, a kit or device for implementing the method for selecting a single-stranded nucleic acid according to the present application. In one embodiment, the kit may be a microplate-based kit.

Single-Stranded Nucleic Acid

Single-Stranded Nucleic Acids Prepared by Method for Selecting Single-Stranded Nucleic Acid According to Present Application

The present application provides a single-stranded nucleic acid prepared by a method for selecting a single-stranded nucleic acid.

In one embodiment, the present application provides a single-stranded nucleic acid having an ability of being bound to a target substance. Furthermore, the single-stranded nucleic acid may have an ability of being bound to two or more target substances. That is, when there are two target substances, the single-stranded nucleic acid may have binding to a first target substance and a second target substance. For example, the target substance may be Brassinolide or Bisphenol A.

In one embodiment, the present application provides a single-stranded nucleic acid having weak binding to a non-target substance. Furthermore, the single-stranded nucleic acid may have weak binding to two or more non-target substances. For example, the non-target substance may be B-sitosterol or Bisphenol S.

In one embodiment, the present application provides a single-stranded nucleic acid having an ability of being bound to a target substance while having weak binding to a non-target substance. In this case, the single-stranded nucleic acid may have an ability of being bound to a target substance and may have relatively weak binding to a non-target substance. Furthermore, the target substance and/or the non-target substance may be of two or more types. For example, the target substance may be Brassinolide and the non-target substance may be B-sitosterol, or the target substance may be Bisphenol A and the non-target substance may be Bisphenol S.

The present application provides a single-stranded nucleic acid consisting of a nucleotide sequence having SEQ ID NO. 1 or 2. In one example, the present application provides a single-stranded nucleic acid having binding to Brassinolide, which consists of a nucleotide sequence of SEQ ID NO. 1, and having relatively weak binding to B-sitosterol. In one example, the present application provides a single-stranded nucleic acid having binding to Bisphenol A, which consists of a nucleotide sequence of SEQ ID NO. 2, and having relatively weak binding to Bisphenol S.

Improvement of Single-Stranded Nucleic Acid

The present application provides an improved single-stranded nucleic acid based on the single-stranded nucleic acid. Through the improvement of the single-stranded nucleic acid, it is possible to prepare a small-sized single-stranded nucleic acid with improved binding to a target substance or similarity to the target substance.

In one example, the improvement of the single-stranded nucleic acids may be performed by truncating a portion of a single-stranded nucleic acid selected by a method for selecting a single-stranded nucleic acid. An improved single-stranded nucleic acid may be prepared by truncating a portion that is mainly bound to a target substance in the single-stranded nucleic acid.

In one example, the improvement of single-stranded nucleic acids may be performed by mutating a portion of a single-stranded nucleic acid selected by a method for selecting a single-stranded nucleic acid.

The present application provides a single-stranded nucleic acid having an ability of being bound to Brassinolide consisting of a nucleotide sequence of SEQ ID NO. 3 or SEQ ID NO. 4 and having relatively weak binding to B-sitosterol.

Use of Single-stranded Nucleic Acid

A single-stranded nucleic acid according to the present application may be used for various purposes. For example, the single-stranded nucleic acid according to the present application may act as an inhibitor against a specific biomolecule. Furthermore, the single-stranded nucleic acid according to the present application may be used for treatment of diseases related to a specific biomolecule. In addition, the single-stranded nucleic acid according to the present application may be used in a sensor, a measurement kit, and the like for detection, quantification, extraction, and purification of a target substance. The present application relates not only a single-stranded nucleic acid, but also to common uses of a single-stranded nucleic acid (or aptamers) using them.

According to the present application, there is provided a pharmaceutical composition for treating a specific disease, the pharmaceutical composition which comprises a single-stranded nucleic acid. In one example, there is provided a pharmaceutical composition for treating a disease related to Bisphenol A, the pharmaceutical composition which includes a single-stranded nucleic acid having binding to Bisphenol A. The pharmaceutical composition may comprise a pharmaceutically acceptable additive. Alternatively, there is provided a method for removing Bisphenol A using a single-stranded nucleic acid having an ability of being bound to Bisphenol A according to the present application.

The present application provides a sensor for detecting a target substance, the sensor which comprising a single-stranded nucleic acid according to the present application. In addition, in the present application, there is provided a kit for quantifying, extracting, or purifying a target substance, the kit which including a single-stranded nucleic acid according to the present application. In one example, there is provided a kit for quantifying, extracting, or purifying Brassinolide, the kit including a single-stranded nucleic acid having an ability of being bound to Brassinolide. In one example, the kit may be a device for selecting a specific target substance, such as chromatography. In one example, the sensor may be a diagnostic kit for diagnosing a specific disease.

### EXAMPLES

### Example 1. Synthesis of Gold Nanoparticles for Test

Gold nanoparticles modified with carboxyl groups were synthesized for verification of protease activity analysis according to the present disclosure.

The gold nanoparticles were synthesized by reduction and stabilization with citrate, as commonly known. In summary, 20 mL of gold tetrachloride (HAuCl4.3H2O, Sigma-Aldrich) with a concentration of 1 mM was added to 50 mL of distilled water and stirred continuously to obtain a solution with a final concentration of 300 nM. 2 mL of 30 mM sodium citrate (C6H5Na3O7.2H2O, Sigma-Aldrich) was added to obtain a final concentration of 600 nM and then stirred. The solution was stirred while boiling so as to be reduced and to form a gold colloid.

### Example 2. Preparation of ssDNA Library for Performing Gold Nanoparticles-Based SELEX (Gold-SELEX)

A random ssDNA library was prepared so that the ratio of A, G, C, and T bases was 1:1:1:1, and primer sites for DNA amplification were positioned at both 3′ and 5′ ends. Gold nanoparticles stabilized with citrate may adsorb most ssDNA but may have poor affinity to some ssDNA depending on the nucleotide composition of the sequence. In order to prevent a sequence having a low binding force to gold nanoparticles from being detached from the gold nanoparticles and selected for a positive SELEX process, regardless of affinity to a target substance, an ssDNA library was prepared in the following two methods at the start of Gold-SELEX.

The first method is to add an additional centrifugation process to the 1st to 4th rounds. This corresponds to Brassinlide Gold-SELEX in the following examples. The composition of 75 ul of gold nanoparticles, 150 nM of ssDNA, and 1× phosphate buffered saline (1×, PBS) is as follows. 0.137M Sodium chloride, 2.7 mM Potassium chloride, 4.3 mM, Sodium phosphate (dibasic, anhydrous), 1.4 mM Potassium phosphate (monobasic, anhydrous)) 20 ul, and distilled water are mixed to obtain a final volume of 190 ul, and then the nanoparticles and ssDNA were left to be bound to each other for 10 minutes. In order to remove an ssDNA having no affinity to the gold nanoparticles, a supernatant was discarded after centrifugation at 6500 g for 10 minutes and the remaining gold nanoparticles and ssDNA were diluted with 190 ul of 0.1×PBS and used for a subsequent SELEX process.

The second method may be performed before proceeding with a first positive SELEX process with a target substance, and the second method corresponds to Bisphenol A Gold-SELEX in the following examples. 75 ul of gold nanoparticles, 150 nM of ssDNA, 20 ul of 1×PBS, and distilled water were mixed to prepare a total sample of 190 ul, and ssDNA and gold nanoparticles were left to bound to each other at room temperature for 10 minutes. Centrifugation was performed at 6500 g for 10 minutes to remove ssDNA not bound to the nanoparticles in the supernatant, and the remaining gold nanoparticles and ssDNA were diluted with 40 ul distilled water. The solution was boiled at 95° C. for 10 minutes to dissociate ssDNA from the nanoparticles, and then centrifuged at 13000 rpm for one minute to obtain only ssDNA from the supernatant. The dissociated ssDNA was amplified through PCR, and the corresponding process was repeated at one to three cycles to remove a sequence not bound to the gold nanoparticles from the ssDNA library, and a prepared library was used for Gold-SELEX.

### Example 3. Monitoring and Aptamer Selection of Gold-SELEX for Brassinolide (Hereinafter Referred to as BL) and Bisphenol A (Hereinafter Referred to as BPA) Based on Colorimetry of Gold Nanoparticles

In order to check the utility of Gold-SELEX, an aptamer was selected for two target substances BL and BPA. Positive SELEX is a SELEX process that increases a binding force between a target substance and ssDNA. 30 pmol of ssDNA library, 75 ul of gold nanoparticles, 1×PBS, and distilled water were mixed to prepare a total of 190 ul of reactants, a reaction was caused at room temperature for ten minutes to allow the gold nanoparticles and ssDNA to be bound, and then a first photographing and absorbance measurement was performed. Every photographing was performed with a camera embedded in a mobile phone in the same place and condition, and absorbance was measured by measuring a wavelength range of 500 to 800 nm in a transparent 96-well plate using a microplate reader. After the measurement, 10 ul of the target substance was added at a desired concentration and a reaction was caused at room temperature for ten to thirty minutes, and the concentration, time, and temperature of this process may vary depending on the type of the target substance and the progress of SELEX. After photographing and absorbance measurement are completed in the same way as in the first method, 10 μl 1M NaCl was added to the sample and a color change was waited for 15 minutes. After the color change was stabilized, the last photographing and absorbance measurement were performed. A photographed sample was transferred to a 1.5 mL microtube and centrifuged at 6500 g for 10 minutes to obtain only a supernatant containing ssDNA and the target substance.

The ssDNA obtained from the supernatant was separated from the target substance through ethanol precipitation. 20μl of 3M sodium acetate and 660μl of cold 100% ethanol (stored at −20° C.) were added to the sample and reacted at −20° C. for 10 minutes. A supernatant was discarded after centrifugation at 14000 rpm for 20 minutes, and 1 mL of cold 70% ethanol was added to wash a ssDNA pellet and additional centrifugation is performed for 15 minutes. After removing all the supernatant and drying the pellet at room temperature for 10 minutes, 40μl distilled water was added to dissolve the DNA. The ssDNA obtained by this process was amplified through polymerase chain reaction (PCR) and used in the next round.

Meanwhile, negative SELEX was also performed to increase specificity of a selected aptamer by reducing a binding force between ssDNA and the counter target substance. The process up to the first photographing and measurement is the same as that of positive SELEX, and 10 ul of the counter target to be excluded instead of the target was mixed with the sample at a desired concentration. Subsequent processes until the third photographing and measurement are the same as in positive SELEX. After the measured sample was transferred to a 1.5 mL microtube and centrifuged at 6500 g for 10 minutes, a supernatant was discarded and 40 ul of distilled water was added to the gold nanoparticles and ssDNA pellet and boiled at 95° C. for 10 minutes to dissociate ssDNA not bound to the counter target substance from the nanoparticles. The dissociated ssDNA was amplified through PCR and used in the next round.

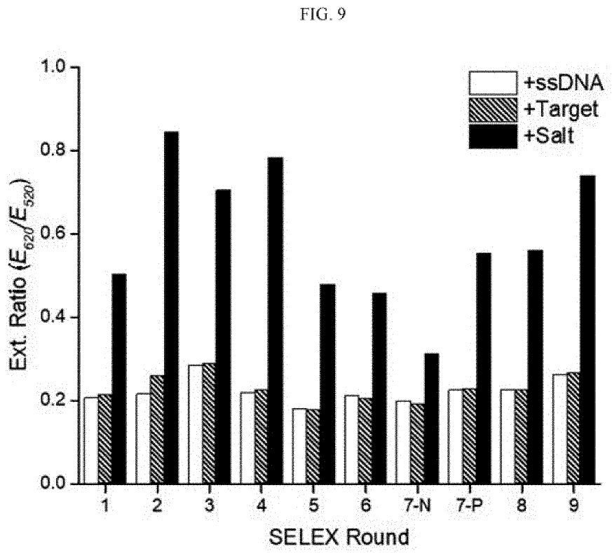

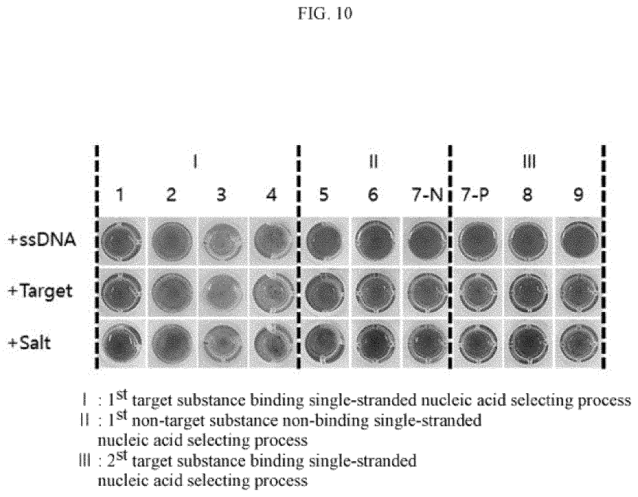

FIGS. 9 and 10 are Gold-SELEX results in which a DNA aptamer that binds to Brassionlide (BL) which is a kind of plant-derived steroid hormone is selected. A total of nine rounds of Gold-SELEX was completed with respect to a substance having no aptamer or antibody and consists of two steps of positive SELEX for selecting a sequence having a high binding force to BL and one step of negative SELEX for selecting a sequence having a low binding force to beta-sitosterol. The structures of a target substance (Brasinolide) and a non-target substance (B-sitosterol) are the same as in Structural Formulas 1 and 2, respectively.

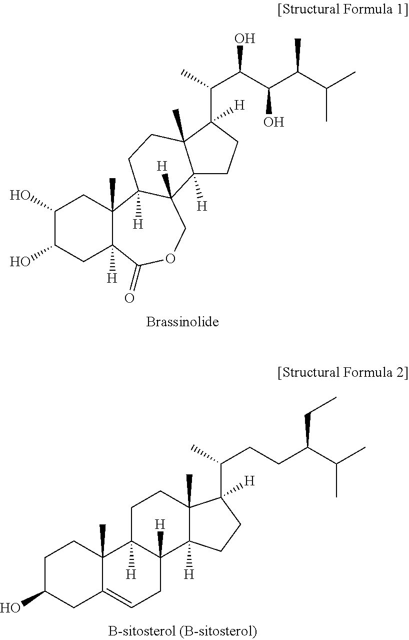

FIGS. 9 and 10 are results of monitoring the entire process of SELEX through color change of gold nanoparticles. The color change was measured as the ratio of an absorbance at 620 nm to an absorbance at 520 nm. The higher the DNA binding force to the target or counter target substance, the more the gold nanoparticles change from red to purple to blue after addition of a salt. As a result, the absorbance at 520 nm decreases and the absorbance at 620 nm increases, so the value of E620/E520 increases.

In FIGS. 9 and 10, the more the positive SELEX for BL progressed until the 1st to 4th rounds, the binding force of the ssDNA pool with respect to the target substance increased, and the color changed from red to purple. During the 5th to 7th rounds, the binding force of the DNA pool with respect to B-sitosterol decreased as the rounds progressed, and the color of the gold nanoparticles changed to red. In the 7th round, it was determined based on the monitoring results that the 6th round product had acquired sufficient target specificity, and the seventh round, which is positive SELEX, was additionally conducted using the same 6th round product, which was called the 7-Pth round. As a result, it was found that the affinity to the target of 7-P was reduced compared to the 4th round, 7-N was discarded and positive SELEX was additionally performed from the 7-Pth to 9th rounds. The SELEX was completed when the binding force to BL was restored again as much as the binding force in the 4th round.

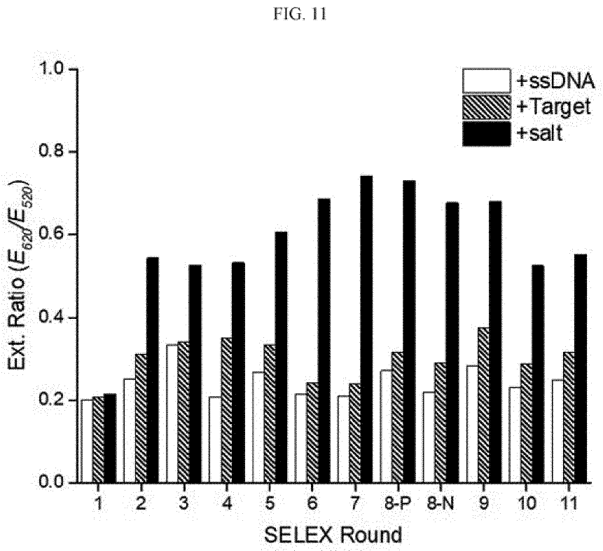

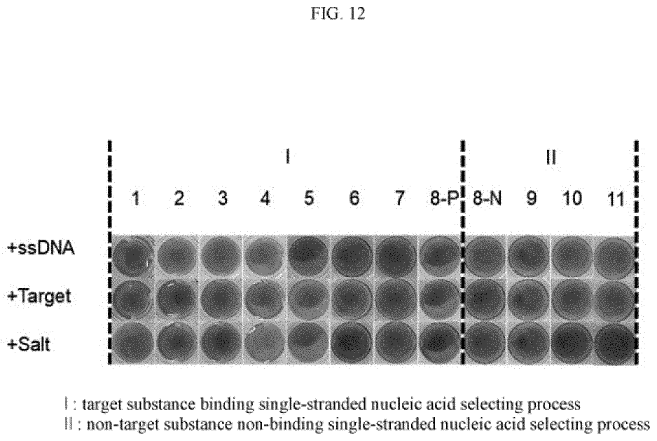

FIGS. 11 and 12 are Gold-SELEX results in which a DNA aptamer bound to Bisphenol A (BPA) (Structure Formula 3), which is a type of endocrine disrupting substance (environmental hormone), is selected. For a known target for which an aptamer has been developed in an existing SELEX method, selection of a new aptamer is completed according to the present disclosure so as to prove the aptamer selection efficiency of Gold-SELEX compared to the existing SELEX method.

A total of eleven rounds of SELEX for BPA were performed and consists of one positive SELEX and one negative SELEX. A target substance and a non-target substance were Bisphenol A (Structural Formula 3) and Bisphenol S (Structural Formula 4), respectively.

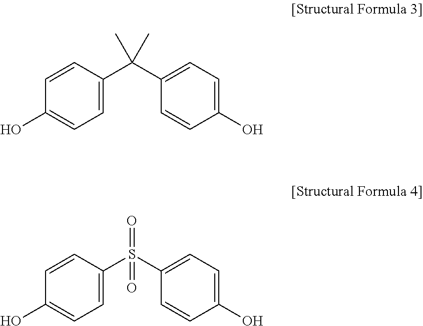 [SMILES: CC(C)(c1ccc(O)cc1)c1ccc(O)cc1 ; O=S(=O)(c1ccc(O)cc1)c1ccc(O)cc1]

FIGS. 11 and 12 are results of monitoring the entire SELEX process using the ratio of an absorbance at 620 nm and an absorbance at 520 nm. All measurements were performed in the same manner as for BL SELEX.

In FIGS. 11 and 12, as positive SELEX for BPA progressed until the 1st to 8th round, the binding force of the ssDNA pool to the target increased, and it was found that the color changed from red to purple. In the 8th round, considering that the DNA pool has obtained sufficient binding to BPA, counter SELEX was additionally performed using Bisphenol S (BPS) at the same concentration as that of the target substance in order to check target specificity of the 7th round product. As a result, it was found that the product of the 7th round product exhibited a binding force to BPS, which is similar to the binding force to BPA. In order to select a BPA aptamer with high target-specific binding force, 8-P was discarded and negative-SELEX was performed from the 8-Nth to 11th rounds. SELEX was completed after the binding force to BPS was reduced as much as the binding force in the 2th round.

### Example 4. Verification of Sequencing Result of Gold-SELEX

After completion of SELEX, for sequence validation, the 9th round product of BL SELEX and the 11 th round product of BPA SELEX were amplified by PCR and undergone through t-vector cloning to be transformed into DH5a competent cells. 50-100 colonies were taken, the plasmid in the colonies was purified and sequenced with the T7 promoter. As a result, sequences with high similarity and high frequency of appearance were mainly analyzed, and the result of the analysis is as shown in the table 2 below.

TABLE 1
List of DNA sequences used in the AuNP-assisted
SELES

| Name | Sequence (5′→3′) |
|---|---|
| Library | ATGCGGATCCCGCGC (N)₃₀₋₄₀ CGCGCGAAGCTTGCG |
| Forward | ATG CGG ATC CCG CGC (SEQ ID NO: 19) |
| primer | |
| Reverse | CGC AAG CTT CGC GCG (SEQ ID NO: 20) |
| primer | |

TABLE 2
Sequencing result

| Target | Group | ID | Frequency | Sequence (N)₃₀₋₄₀ |
|---|---|---|---|---|
| Brassinotides | 1 | #BLA9-20 | 4 | CGT GCA GAG GGA GAC CGG TAC CCG TTC GTG (SEQ ID NO: 1) |
| | 2 | #BLA9-11 | 2 | TCC GTG AGA CGG CAA ATT ATG GGT TAT ATG (SEQ ID NO: 2) |
| | 3 | #BLA9-34 | 2 | CCA GAA CAT CAT CCC GGG TTC TAA TTT GTG (SEQ ID NO: 3) |
| | 4 | #BLA9-3 | 2 | CGA GGA TAT AGA GCT ACA GTT AAT AAT GGG (SEQ ID NO: 4) |
| Bisphenol A | 1 | #nBPA53 | 3 | CCA AAA GTT TAA GCG CGA AGA TAC TGT TGC GTC CAC GGG C |
| | | | | (SEQ ID NO: 5) |
| | | #nBPA36 | | CCA GAA GTT AAA GCG CGA AGA TAC TGT TGC GTC CAC GGG C |
| | | | | (SEQ ID NO: 6) |
| | 2 | #nBPA20 | 6 | CCC AAT TGA AGA ACG CGC GAA GAA TAT AAG GTG GCC TGG C |
| | | | | (SEQ ID NO: 7) |
| | | #nBPA40 | | CCC AAC TGA AGG ACG CGC GAA GAA TAT AAG GTG GCC TGG C |
| | | | | (SEQ ID NO: 8) |
| | | #nBPA32 | | CCC AAC TGA GAA ACG CGC GAA GAA TAT AAG GTG GCC TGG C |
| | | | | (SEQ ID NO: 9) |
| | | #nBPA37 | | CCC AAC TTA GAA GCG CGC GAA GAA TAT AAG GTG GCC TGG C |
| | | | | (SEQ ID NO: 10) |
| | | #nBPA54 | | CCC AAC TGA GGA ACG CGC GAA GAA TAT AAG GTG GCC TGG C |
| | | | | (SEQ ID NO: 11) |
| | | #nBPA21 | | CCC AAC TGA AGA ACG CGC GAA GAA TAT AAG GTG GCT TGG C |
| | | | | (SEQ ID NO: 12) |
| | | #nBPA50 | | CCC AAC TGA GAA ACG CGC GAA GAA TAT AAG GTG GCT TGG C |
| | | | | (SEQ ID NO: 13) |
| | 3 | #nBPA19 | 11 | CCA ACG GAG GAC TAT TAA GCG CGA AGG TGG CGG TAT TGT G |
| | | | | (SEQ ID NO: 14) |
| | | #nBPA67 | | CCA ACG GAG GAC TAT TAA GCG CGA AGA TGG CGG TAT TGT G |
| | | | | (SEQ ID NO: 15) |
| | 4 | #nBPA42 | 3 | GCG AAG TAT ACA GTT AGG CCG TGT GTT GGC (SEQ ID NO: 16) |

### Example 5. Verification of Aptamer's Binding Force and Specificity to BL and BPA Based on Colorimetry of Gold Nanoparticles

From the product of each final SELEX round, one sequence having highest affinity to a target was selected, and target binding and specificity were verified for each candidate. All experiments were conducted under the same conditions as Gold-SELEX.

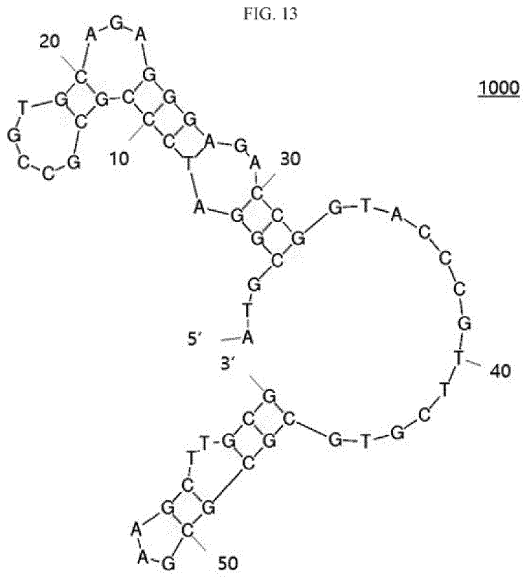

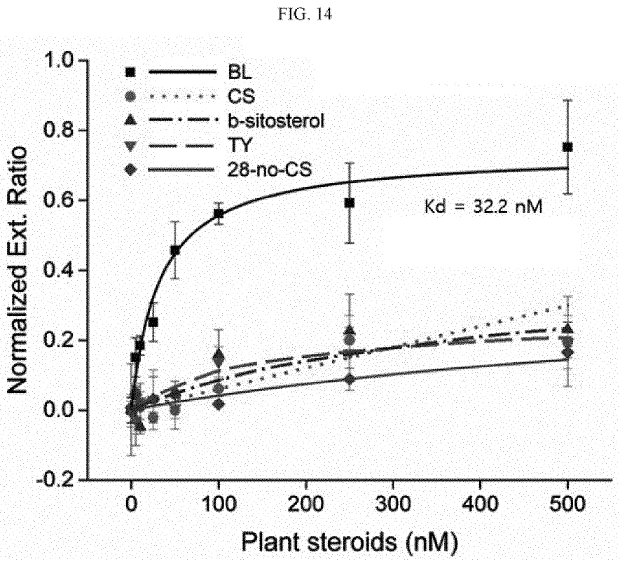

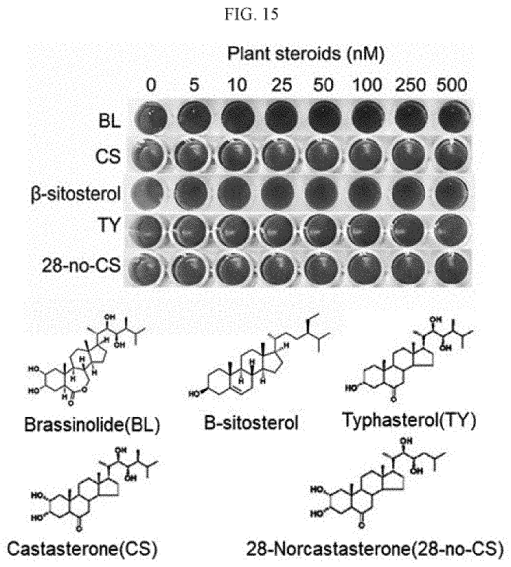

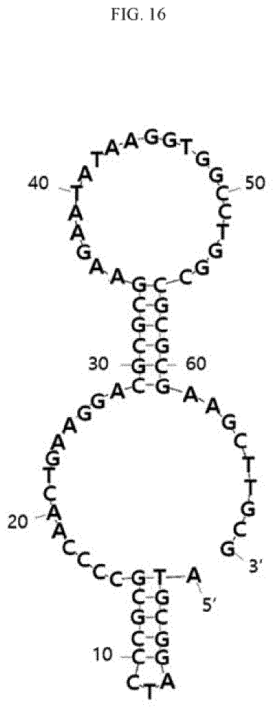

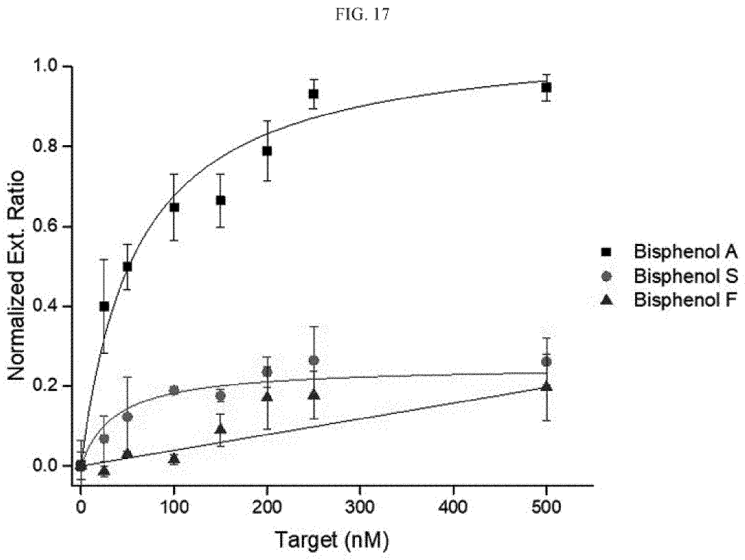

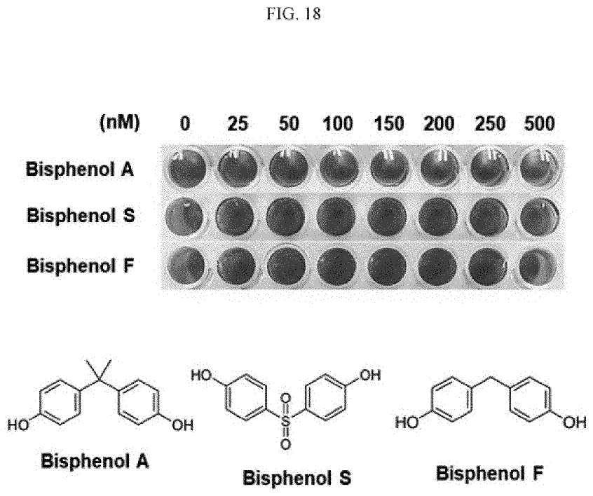

FIGS. 13 to 15 are results that verifies the binding force and specificity of an aptamer selected for BL with respect to target substances and counter target substances, and FIGS. 16 to 18 are results that verifies the binding force and specificity of an aptamer selected for BPA with respect to target substances and counter target substances. FIG. 13 is a secondary structure of BLA9-20 which is a finally selected BL aptamer, and FIG. 16 is a secondary structure of nBPA40 which is a finally selected BPA aptamer. Both structures were predicted and selected as the sequence with the highest thermodynamic stability from the Mfold web server (M. Zuker. Mfold web server for nucleic acid folding and hybridization prediction, Nucleic Acids Res. 31 (13), 3406-3415, 2003.). In addition, it may be found that the aptamers selected from FIGS. 14, 15, 17, and 18 have the highest binding force to respective target substances and have the relatively low binding force to other substances of similar structures. For accurate comparison, the absorbance ratio E620/E520 measured in FIGS. 14 and 17 was normalized into a formula of ((Ext Ratio with analyte)−(Ext. Ratio without analyte))/(Ext. Ratio without analyte). Standard reagents were diluted at each concentration before experiments and then diluted in 100% ethanol. The structures of the substances used in the experiments of FIGS. 14 and 15 are shown at the bottom of FIG. 15, and all the substances are intermediate products of the process of BL biosynthesis in plants. The types and structures of the substances used in FIGS. 17 and 18 are shown at the bottom of FIG. 18.

### Example 6. Comparison of a Known BPA Aptamer and a BPA Aptamer Selected by Gold-SELEX

A BPA DNA aptamer selected by another SELEX method and a new aptamer sequence selected by Gold-SELEX according to the present application were compared in target detection capability through colorimetry of gold nanoparticles, and the nucleotide sequences of the two aptamers were checked by a multi-alignment method.

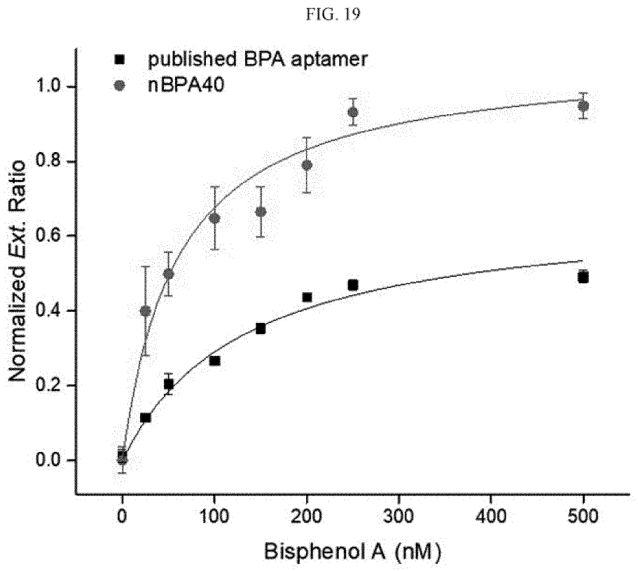

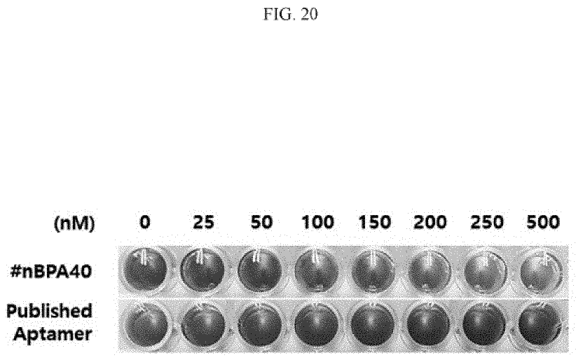

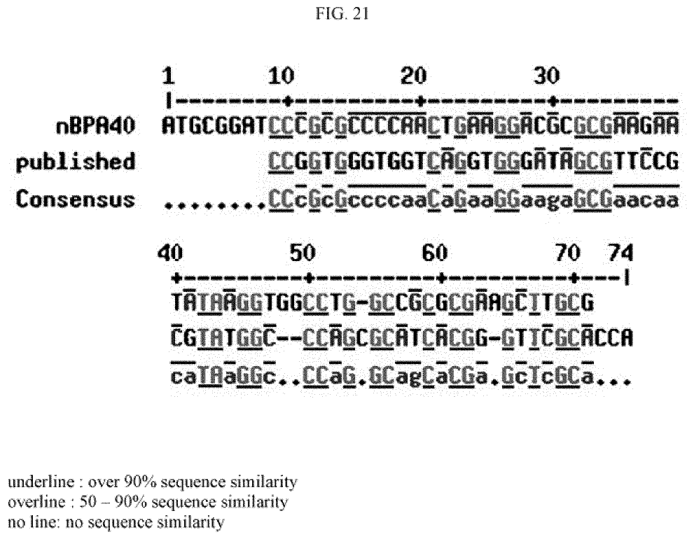

Experimental conditions were the same as in the aptamer selecting process of Gold-SELEX, and as can be seen from the results of FIGS. 19 and 20, it seems that nBPA40, which is the aptamer selected by Gold-SELEX, is more suitable for use as a gold nanoparticle colorimetric sensor than the previously reported aptamer. In addition, in spite of being selected by other methods, a high similarity between nBPA40 and the existing aptamer was found in the result of FIG. 21. Red indicates similarity of 90% or higher, blue indicates similarity of 50 to 90%, and black indicates similarity less than 50%.

### Example 7. Improvement of Brassinolide-Binding Aptamer

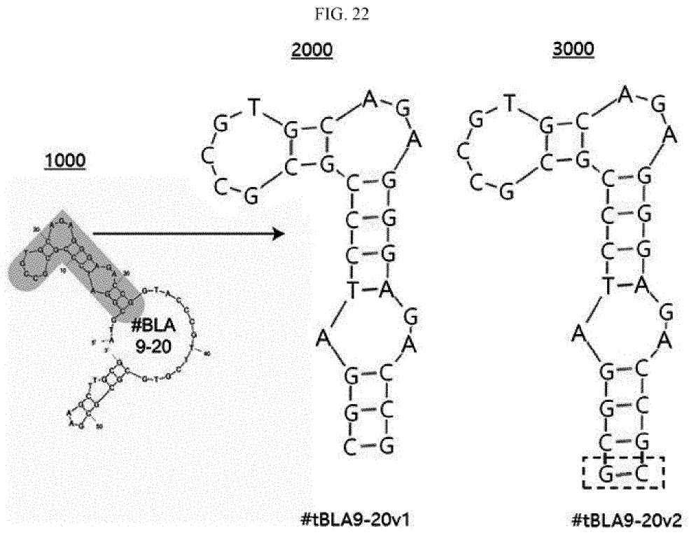

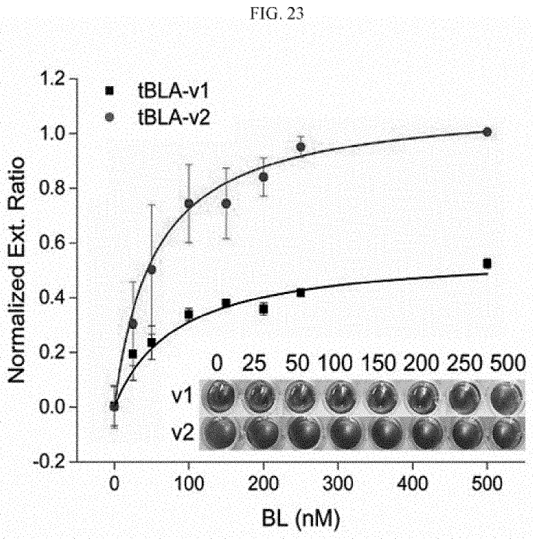

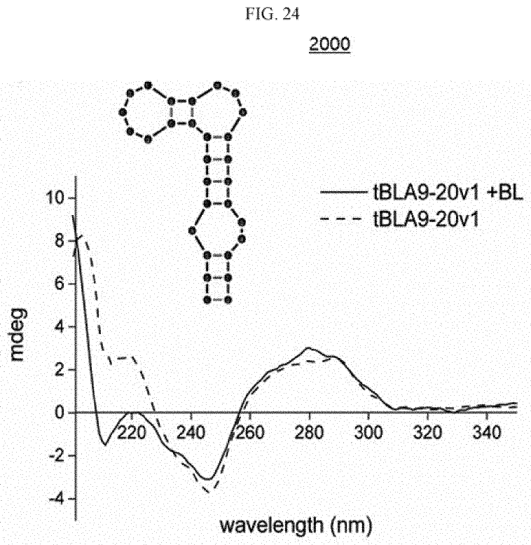

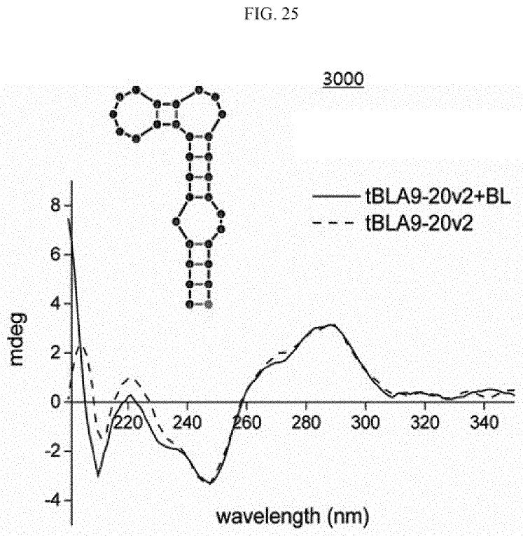

FIGS. 22 to 25 are results of comparison of a binding force to Brassinolide by improving BLA9-20 aptamer. As shown in FIG. 22, a truncated aptamer (tBLA9-20v1, total 29 bp, SEQ ID NO. 17) formed by cutting only the marked portion in BLA9-20 (total of 60 bp) and a truncated aptamer (tBLA9-20v2, total 31 bp, SEQ ID NO. 18) formed by adding a single base pair were prepared. In a result of measuring the binding force of these two truncated aptamers to Brassinolide through colorimetry of gold nanoparticles, the binding force of BLA9-20v2 was measured relatively higher than that of tBLA9-20v1, and the binding affinity of tBLA9-20v2 was slightly higher (Kd=54.2 nM) than that of BLA9-20 (FIG. 23). In a result of comparing the secondary structures of tBLA9-20v1 and tBLA9-20v2 using circular dichroism (CD) before and after treatment of Brassinolide, it was found that there is change in the secondary structure of tBLA9-20v1 whereas there is no change in the secondary structure of tBLA9-20v2 (FIGS. 24 and 25).

### Example 8. Method for Detection of Brassinolide Using Brassinolide Binding Aptamer

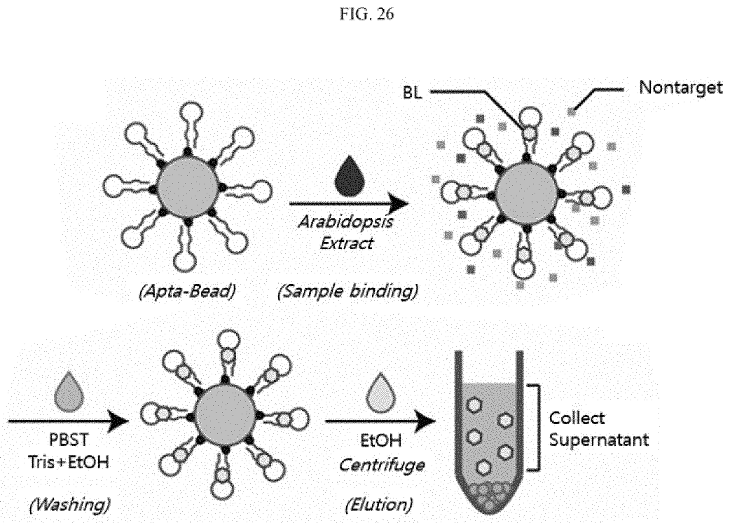

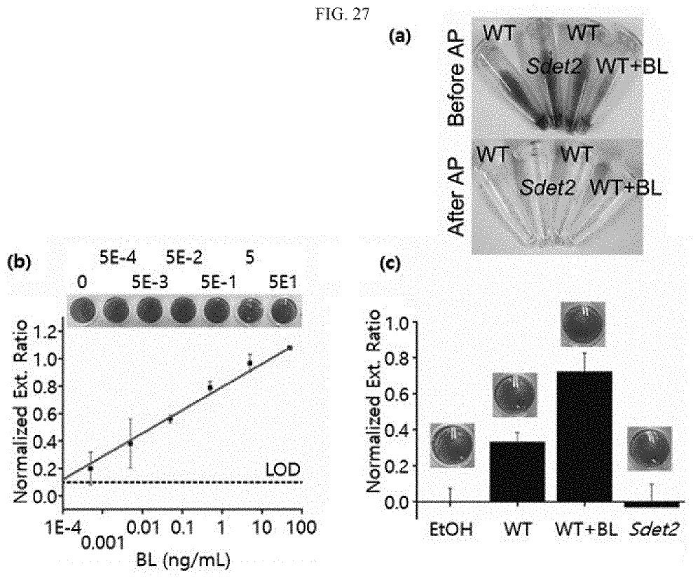

FIG. 26 illustrates that aptamer precipitation, which is similar to immunoprecipitation using an antibody, can be used as a method for detecting Brassinolide using an aptamer. That is, the surface of microbead coated with avidin may be bound to biotin-aptamer (biotin-tBLA9-20v2), reacted with Arabidopsis extract containing Brassinolide at a different concentration, and then washed. The Brassinolide contained in the Arabidopsis extract is strongly bound to the surface of microbead, and after treatment with ethanol, Brassinolide bound to an aptamer may be effectively extracted and quantified through colorimetry of gold nanoparticles. In FIG. 27, real wild-type Arabidopsis extract (WT, wild-type Arabidopsis), mutant Arabidopsis extract (Sdet2, BL-deficient mutant Arabidopsis) grown in a medium in which Brassinolide biosynthesis is inhibited, and Arabidopsis extract grown in a media in which Brassinolides is rich in the wild-type Arabidopsis (WT+BL, wild-type Arabidopsis with BL-rich media) were analyzed and compared with a standard calibration curve. It was found that Sdel2 is relatively less compared to the wild-type Arabidopsis, and WT-BL exhibits a very high color range, verifying that quantitative analysis was possible.

### DETAILED DESCRIPTION OF REFERENCE NUMERALS

110, 210, 310, 410: preparing a gold nanoparticle-single-stranded nucleic acid library
111: preparing a gold nanoparticle
112: preparing a single-stranded nucleic acid library
113: reacting the gold nanoparticles with a single-stranded nucleic acid library
120, 220, 320, 420: reacting the gold nanoparticle-single-stranded nucleic acid library with a target substance
130, 230, 330, 430: separating the single-stranded nucleic acid bound to the target substance from the reaction mixture
240: isolating the single-stranded nucleic acid after separating the single-stranded nucleic acid and the target substance
250: amplifying the isolated single-stranded nucleic acid
340: determining whether to proceed with an additional reaction through the color index of the reaction mixture
340′: comparing the color index of the reaction mixture to a standard color index
350: additional process
350′: additional process—repeating a target substance binding single-stranded nucleic acid selecting process with respect to the same target substance
510: preparing a gold nanoparticle-single-stranded nucleic acid library after performing a target substance binding single-stranded nucleic acid selecting process
520: non-target substance non-binding single-stranded nucleic acid selecting process
1000: aptamer BLA9-20
2000: improved aptamer tBLA9-20v1
3000: improved aptamer tBLA9-20v2

1. A method for selecting a single-stranded nucleic acid having an ability of being bound to a target substance, the method comprising:
i) preparing a gold nanoparticle-single-stranded nucleic acid library;
ii) reacting the gold nanoparticle-single-stranded nucleic acid library with a target substance;
iii) separating the single-stranded nucleic acid bound to the target substance from the reaction mixture; and
iv) determining whether to proceed with an additional reaction through the color index of the reaction mixture.

2. The method of claim 1,
Wherein preparing the gold nanoparticle-single-stranded nucleic acid library comprises:
a) preparing gold nanoparticles having an average diameter of 15 nm to 50 nm by reduction and stabilization with citrate;
b) preparing a single-stranded nucleic acid library;
c) reacting the gold nanoparticles with the single-stranded nucleic acid library; and
d) removing the single-stranded nucleic acid not bound to the gold nanoparticles.

3. The method of claim 1,
Wherein separating the single-stranded nucleic acid bound to a target substance from the reaction mixture is performed by obtaining a supernatant after centrifuging the reaction mixture.

4. The method of claim 1, the method further comprising:
isolating the single-stranded nucleic acid from the target substance after separating the single-stranded nucleic acid bound to the target substance from the reaction mixture.

5. The method of claim 4,
Wherein isolating the single-stranded nucleic acid is performed by ethanol precipitation.

6. The method of claim 1, the method further comprising:
amplifying the isolated single-stranded nucleic acid after separating the single-stranded nucleic acid bound to the target substance.

7. The method of claim 1,
wherein determining whether to proceed with an additional reaction through the color index of the reaction mixture comprises:
a) measuring a color index of the reaction mixture; and
b) comparing the color index of the reaction mixture to a standard color index.

8. The method of claim 7, the method further comprising:
inducing the gold nanoparticles to aggregate prior to measuring the color index of the reaction mixture.

9. The method of claim 8,
wherein inducing the gold nanoparticles to aggregate is carried out adding a salt to the reaction mixture.

10. The method of claim 1,
Wherein the color index of the reaction mixture is quantified as the ratio of the absorbance values measured at two wavelengths before the gold nanoparticles are aggregated and the absorbance values measured at the same two wavelengths after the gold nanoparticles are aggregated.

11. The method of claim 1, the method further comprising:
performing sequentially separating the single-stranded nucleic acid bound to the target substance from the reaction mixture after determining whether to proceed with an additional reaction through the color index of the reaction mixture.

12. The method of claim 1,
wherein the target substance comprises Brassinolide or a small molecule material capable of inducing aggregation of gold nanoparticles including Bisphenol A, ions, proteins, nucleic acids, viruses, and microorganisms.

13. The method of claim 1, the method further comprising:
a) separating a single-stranded nucleic acid bound to a target substance from the reaction mixture;
b) after determining whether to proceed with an additional reaction through the color index of the reaction mixture, preparing a gold nanoparticle-single-stranded nucleic acid library of single-stranded nucleic acids bound to the target substance;
c) reacting the gold nanoparticle-single-stranded nucleic acid library with a non-target substance; and
d) separating the single-stranded nucleic acids that are not bound to the non-target substance from the reaction mixture;
wherein the target substance and the non-target substance are different.

14. An isolated single-stranded nucleic acid consisting of one nucleotide sequence selected from SEQ ID NOs: 1, 17 and 18, and having an ability of being bound to Brassinolide and relatively weak binding to B-sitosterol.

15. A kit for purifying Brassinolide comprising a single-stranded nucleic acid consisting of one or more nucleotide sequences selected from SEQ ID NOs: 1, 17 and 18.
